(19) United States
(12) Patent Application Publication
(10) Pub. No.: US 2014/0022277 A1
(43) Pub. Date: Jan. 23, 2014

(54) MEDICAL IMAGE GENERATION APPARATUS AND MEDICAL IMAGE MANAGEMENT SYSTEM

(71) Applicant: KONICA MINOLTA, INC., Tokyo (JP)

(72) Inventors: Osamu TAKEDA, Tokyo (JP); Katsumi HOSOKAWA, Tokyo (JP); Ken INOUE, Tokyo (JP)

(21) Appl. No.: 13/937,503

(22) Filed: Jul. 9, 2013

(30) Foreign Application Priority Data
Jul. 19, 2012 (JP) 2012-160091

(51) Int. Cl. A61B 5/00

(52) U.S. Cl. CPC A61B 5/0033
USPC 345/619

(57) ABSTRACT

A medical image generation apparatus is communicably connected to an information processing apparatus which receives inputting and performs displaying of information regarding a test object. The medical image generation apparatus includes: an image data generation section to generate image data of the examination region of the test object; and a control section to request the information processing apparatus to transmit test-object specifying information for specifying the test object, to receive the test-object specifying information transmitted from the information processing apparatus, to obtain test-object information corresponding to the received test-object specifying information from a predetermined test-object information storage section, and to correlate the obtained test-object information to the image data created by the image data generation section.

### BACKGROUND OF THE INVENTION

1. Field of the Invention

The present invention relates to a medical image generation apparatus and a medical image management system.

2. Description of Related Art

Heretofore, when performing a diagnosis of a medical image by using a medical image generation apparatus such as an ultrasound image diagnosis device, an operator including a doctor and a technician reads a medical chart of a patient as a test object, manually inputs various pieces of information regarding the patient such as ID, name and age of the patient directly in the apparatus, and then takes an image of examination region of the patient.

Thus manually inputting the various pieces of information regarding the patient every time the image is taken using the apparatus is a very troublesome job for the operator and likely causes an inputting error.

Japanese Patent Application Laid-open No. 2010-274067 discloses an ultrasound diagnosis device which receives delivery of a list of patient data, namely a list of patients to be examined, from an information processing apparatus such as a management server, obtains the patient data, namely detailed data of the patient, from the beginning of the examination so that the examination is performed in the order shown in the list of patient data, and sets the obtained patient data to conduct management of the examination. At that time, in the case of performing the examination on an arbitrary patient among the patient shown in the list of patient data, the operator conducts an operation to select the arbitrary patient.

In an actual medical scene, for example, an image is sometimes taken by using the medical image generation apparatus in an examination room after a doctor conducts a medical interview there. Especially the ultrasound image diagnosis device is often used easily just like using a stethoscope. In the technique described in Japanese Patent Application Laid-open No. 2010-274067, however, it is necessary to find out the intended patient from the list of patient data for setting the patient data even in such case. Such inputting operation is very troublesome.

### SUMMARY OF THE INVENTION

In view of the foregoing, an object of the present invention is to provide a medical image generation apparatus and a medical image management system which can set information on a patient with accuracy and which are implementable by simple operations.

In order to achieve the above-described object, the medical image generation apparatus in which a first aspect of the present invention is reflected is communicably connected to an information processing apparatus which receives inputting and performs displaying of information regarding a test object, and the medical image generation apparatus includes: an image data generation section to generate image data of the examination region of the test object; and a control section to request the information processing apparatus to transmit test-object specifying information for specifying the test object, to receive the test-object specifying information transmitted from the information processing apparatus, to obtain test-object information corresponding to the received test-object specifying information from a predetermined test-object information storage section, and to correlate the obtained test-object information to the image data created by the image data generation section.

Preferably, the medical image generation apparatus further includes an operation section, and the control section requests the information processing apparatus to transmit the test-object specifying information depending on a predetermined operation in the operation section.

Preferably, in the medical image generation apparatus, the control section requests an electronic medical chart terminal as the information processing apparatus to transmit the test-object specifying information, the electronic medical chart terminal being used to create medical record data of the test object, and receives the test-object specifying information from the electronic medical chart terminal.

Preferably, in the medical image generation apparatus, the control section requests a receipt terminal as the information processing apparatus to transmit the test-object specifying information, the receipt terminal being used to create receipt information, and receives the test-object specifying information from the receipt terminal.

Preferably, in the medical image generation apparatus, the control section requests a medical image management apparatus as the information processing apparatus to transmit the test-object specifying information, the medical image management apparatus being used to manage the image data to which accessory information is attached, and receives the test-object specifying information from the medical image management apparatus.

Preferably, in the medical image generation apparatus, the image data generation section outputs transmission ultrasound wave toward the test object, receives reflective ultrasound wave from the test object to obtain a receiving signal, and creates ultrasound image data on the basis of the obtained receiving signal.

### BRIEF DESCRIPTION OF THE DRAWINGS

The present invention will become more fully understood from the detailed description given hereinbelow and the appended drawings, and thus are not intended as a definition of the limits of the present invention, and wherein:

FIG. 1 is a system configuration diagram of a medical image management system according to this embodiment;

FIG. 2 is a diagram illustrating an example of arrangement of apparatuses in a medical facility when applying the medical image management system according to the embodiment thereto;

FIG. 3 is a block diagram illustrating functional configurations of an electronic medical chart terminal, a receipt terminal, a PACS terminal, an image server, and a medical examination information server;

FIG. 4 is a block diagram illustrating a functional configuration of an ultrasound image diagnosis device;

FIG. 5 is a block diagram illustrating a functional configuration of a control section of the ultrasound image diagnosis device;

FIG. 6 is a flowchart for explaining about default setting processing;

FIG. 7 is a flowchart for explaining about ultrasound diagnostic processing;

FIG. 8 is a flowchart for explaining about temporary patient information generating processing;

FIG. 9 is a flowchart for explaining about ultrasound wave transmitting/receiving processing;

FIG. 10 is a flowchart for explaining about the ultrasound wave transmitting/receiving processing;

FIG. 11 is a flowchart for explaining about patient-to-be-displayed identification information transmitting processing;

FIG. 12 is a flowchart for explaining about patient information transmitting processing;

FIG. 13 is a flowchart for explaining about image referring processing;

FIG. 14 is a diagram for explaining about a patient information screen;

FIG. 15 is a diagram for explaining about a display image to be displayed in a display section;

FIG. 16 is a diagram for explaining about display contents to be displayed in a basic information display region;

FIG. 17 is a diagram for explaining about the display contents to be displayed in the basic information display region;

FIG. 18 is a diagram for explaining about the display screen to be displayed in the display section;

FIG. 19 is a system configuration diagram of a medical image management system according to a second embodiment;

FIG. 20 is a system configuration diagram of a medical image management system according to a third embodiment;

FIG. 21 is a block diagram illustrating a functional configuration of a control section of a conventional ultrasound image diagnosis device;

FIG. 22 is a block diagram illustrating a functional configuration of a control section of a ultrasound image diagnosis device according to another embodiment; and

FIG. 23 is a diagram for explaining about received-data-from-external-inputting-apparatus definition information.

### PREFERRED EMBODIMENTS OF THE PRESENT INVENTION

Hereinafter, a medical image management system according to an embodiment of the present invention will be described with reference to the drawings. In this regard, however, the scope of the present invention is not limited to illustrated examples. In the following descriptions, the same functions/configurations are designated by the same numerals, and the descriptions thereof are omitted.

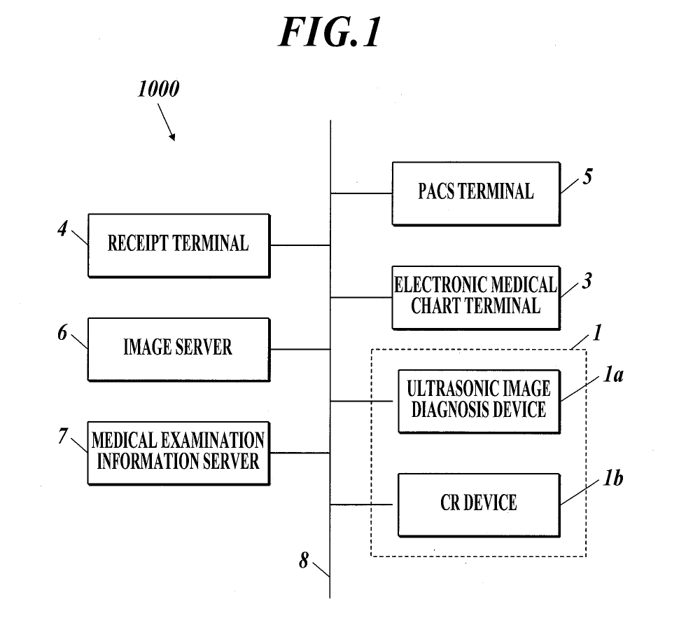

As illustrated in FIG. 1, a medical image management system 1000 is configured to include: an ultrasound image diagnosis device 1a and a Computed Radiography (CR) device 1b which constitute a medical image generation apparatus 1; an electronic medical chart terminal 3; a receipt terminal 4; a Picture Archiving and Communication System (PACS) terminal 5 as a medical image management apparatus; an image server 6; and a medical examination information server 7, and those apparatuses/devices are connected to one another via a Local Area Network (LAN) 8, for example.

As a communication system used in a hospital, the Digital Imaging and COmmunication in Medicine (DICOM) standard is generally used, and the DICOM Modality Worklist Management (DICOM MWM) and the DICOM Modality Performed Procedure Step (DICOM MPPS) are used in communication between the apparatuses/devices connected to one another via the LAN. The communication system adaptable to the embodiment is not limited to the abovementioned examples.

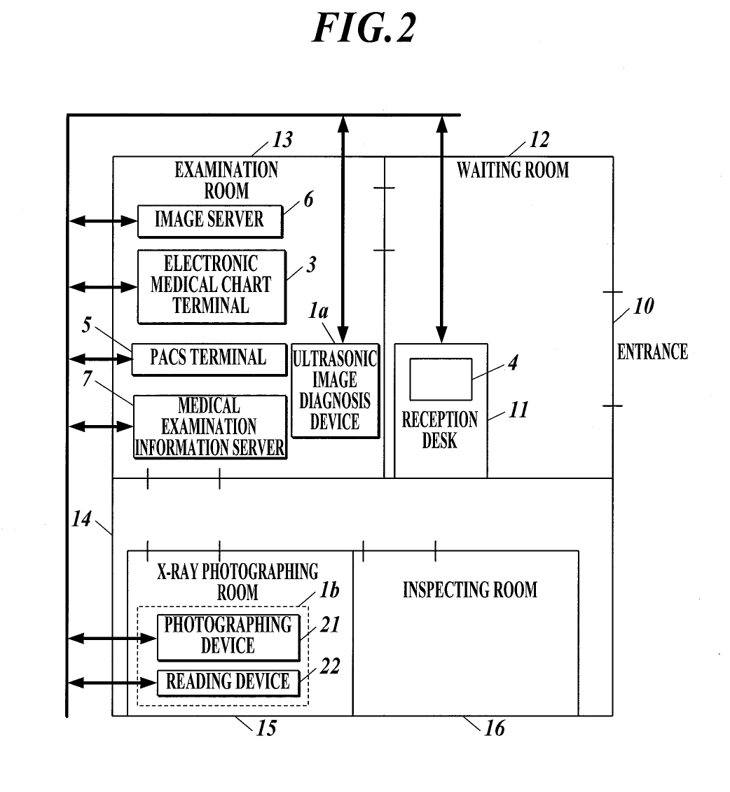

For example, in a small-scale facility such as an office of a practitioner and a clinic, the apparatuses/devices are arranged as shown in FIG. 2.

Concretely, a patient who enters an entrance 10 finds a reception desk 11 and a waiting room 12. A receptionist is disposed at the reception desk 11. The receptionist gives the patient, who has come to the office or clinic, a number plate(s) on which a receipt number (for example, a serial number given according to the order of arrival of the day) is printed, which number is used for distinguishing individual patient according to the order of arrival. The receipt terminal 4 is set on the reception desk 11, which terminal 4 is used for calculation of insurance points, accounting calculation, and so on. The receptionist refers to a medical card or the like possessed by the patient to input the receipt number, a name of patient, an ID of patient, and the like to the receipt terminal 4 while correlating these pieces of information one another. The receipt terminal 4 requests the medical examination information server 7 to transmit an electronic medical chart as medical record data in which detailed information on the patient is written, which information corresponds to the input data such as the name of patient and the ID of patient. After the receipt terminal 4 receives the electronic medical chart from the medical examination information server 7, the receptionist confirms contents of the electronic medical chart on a display screen of the receipt terminal 4 and instructs the receipt terminal 4 to transmit the electronic medical chart to the electronic medical chart terminal 3. Then the receipt terminal 4 transmits the electronic medical chart of the patient who has come to the office/clinic to the electronic medical chart terminal 3. After medical examination on the patient, the receptionist inputs necessary information regarding accounting and receipt (accounting calculation, calculation of insurance points for insurance claim, and so on) to the receipt terminal 4 on the basis of the information written in the electronic medical chart. When completing the input of the information regarding receipt, the receptionist confirms payment for the medical examination displayed on the display screen of the receipt terminal 4, and then receives the payment from the patient and issues a receipt for the medical examination to provide the patient with the receipt.

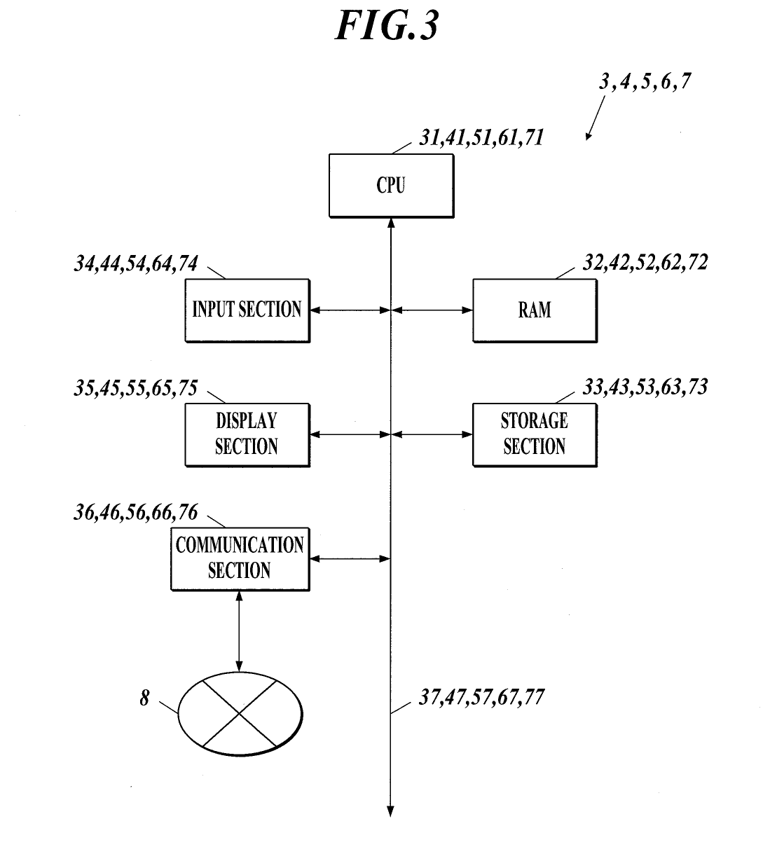

In the next room to the waiting room 12, there is an examination room 13 in which a doctor conducts medical examination and/or diagnostication, with a door between the waiting room 12 and the examination room 13. On a desk (not illustrated) for the medical examination in the examination room 13, the electronic medical chart terminal 3 and the PACS terminal 5 are disposed, and the image server 6, the medical examination information server 7, and the ultrasound image diagnosis device 1a are arranged in predetermined positions in the examination room 13. The electronic medical chart terminal 3 stores a plurality of patients to be examined in the order of receiving the electronic medical chart from the receipt terminal 4. Thus, each of the patients is put in a waiting state. During this time, the patient waits in the waiting room 12. The electronic medical chart terminal 3 displays the content of the electronic medical chart of the patient to be examined next in a display section 35 (see FIG. 3), when the doctor performs operation for updating the patient to be examined. Then the doctor allows the patient who is examined next to enter the examination room 13, and starts the medical examination. The doctor conducts the medical examination and inputs a content/result of the medical examination to the electronic medical chart terminal 3. The doctor performs the medical examination by using the ultrasound image diagnosis device 1a as needed basis. The ultrasound image diagnosis device 1a saves ultrasound image data obtained as the result of the medical examination in the image server 6. The image server 6 is equipped with an image Data Base (DB) to store image data such as ultrasound image data. The image data stored in the image server 6 is read out by the PACS terminal 5 so that the medical image is displayed in a display screen of the PACS terminal 5. The doctor provides the patient with an explanation by using the medical image displayed in the display screen of the PACS terminal 5, and inputs findings in the electronic medical chart terminal 3. When the medical examination is terminated and the doctor inputs an instruction to terminate the medical examination to the electronic medical chart terminal 3, the electronic medical chart terminal 3 transmits the electronic medical chart to the receipt terminal 4. After that, the patient leaves the examination room 13 to return to the waiting room 12. The doctor conducts the operation for updating the patient to be examined in the above-described way, and allows the patient who is examined next to enter the examination room 13 to perform the medical examination.

Moreover, an X-ray photographing room 15 for performing X-ray photography is located on the opposite side of the examination room 13 across a corridor 14. In the X-ray photographing room 15, a CR device 1b which is constituted by a photographing device 21 and a reading device 22 is disposed. Next to the X-ray photographing room 15, there is an inspecting room 16.

Next, functional configurations of the electronic medical chart terminal 3, the receipt terminal 4, the PACS terminal 5, the image server 6, and the medical examination information server 7 according to the embodiment will be described. Since the electronic medical chart terminal 3, the receipt terminal 4, the PACS terminal 5, the image server 6, and the medical examination information server 7 have the same configurations as one another, the electronic medical chart terminal 3 will be described herein and explanations for other devices are omitted.

As shown in FIG. 3, the electronic medical chart terminal 3 is configured to include: a CPU 31; a RAM 32; a storage section 33; an input section 34; the display section 35; a communication section 36, etc. The respective sections are connected to one another via a bus 37.

The CPU 31 reads out various programs such as system programs and processing programs stored in the storage section 33 to expand the read programs in the RAM 32, and centrally controls the respective sections of the electronic medical chart terminal 3 according to the expanded programs.

The RAM 32 forms a work area to temporarily store the various programs read out from the storage section 33 and executable by the CPU 31, input/output data, parameters, and so on, in various processes executed by the CPU 31.

The storage section 33 is composed of a Hard Disk Drive (HDD), a nonvolatile semiconductor memory, or the like. The storage section 33 stores the system program and/or the various programs to be executed by the CPU 31.

The input section 34 is composed of a keyboard which includes cursor keys, numeral input keys, various function keys, etc., and a pointing device such as a mouse, etc. The input section 34 outputs to the CPU 31a depressed key signal obtained by key depressing operation, and operation signal obtained by mouse operation, as input signals.

The display section 35 is configured to include a monitor such as a Liquid Crystal Display (LCD) and a Cathode Ray Tube (CRT), for example, and displays various screens according to instructions of display signals input from the CPU 31.

The communication section 36 is composed of network interface and the like, and performs transmission/reception of data between the electronic medical chart terminal 3 and an external device connected to the LAN 8.

Next, a functional configuration of the ultrasound image diagnosis device 1a according to the embodiment will be described.

The ultrasound image diagnosis device 1a is a device which displays/outputs a state of internal biological tissue of the patient (hereinafter sometimes called as a test object) as the ultrasound image. Concretely, the ultrasound image diagnosis device 1a transmits ultrasound wave (transmission ultrasound wave) into the test object such as a biological body, and receives a reflected wave (reflective ultrasound wave: echo) of the ultrasound wave which is reflected inside the test object. The ultrasound image diagnosis device 1a converts the received reflective ultrasound wave into an electrical signal, and creates the ultrasound image data on the basis of this electrical signal. The ultrasound image diagnosis device 1a then displays the internal state of the test object as the ultrasound image on the basis of the created ultrasound image data. The ultrasound image diagnosis device 1a also creates accessory information regarding the created ultrasound image data on the basis of the patient information which is of the information regarding the patient, and the like. The ultrasound image diagnosis device 1a can create an image file composed of DICOM image data compliant with the DICOM standard by attaching the accessory information to the ultrasound image data.

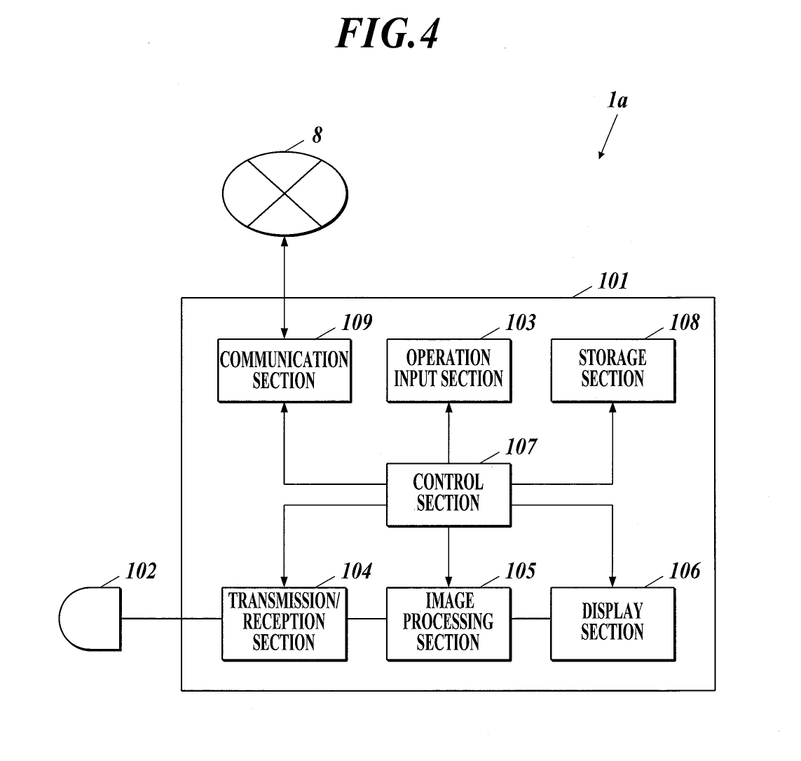

As shown in FIG. 4, the ultrasound image diagnosis device 1a includes an ultrasound image diagnosis device body 101 and an ultrasound probe 102. The ultrasound probe 102 transmits the transmission ultrasound wave into the test object, and receives the reflective ultrasound wave from the inside of the test object. The ultrasound image diagnosis device body 101 is connected to the ultrasound probe 102 via a cable, and transmits a driving signal of the electric signal to the ultrasound probe 102 so that the ultrasound probe 102 transmits the transmission ultrasound wave into the test object. The ultrasound image diagnosis device body 101 also receives a receiving signal which is the electric signal created in the ultrasound probe 102 correspondingly to the reflective ultrasound wave received thereby from the inside of the test object, and creates the ultrasound image data as described above.

The ultrasound image diagnosis device body 101 is configured to include an operation input section 103, a transmission/reception section 104, an image processing section 105, a display section 106, a control section 107, a storage section 108, and a communication section 109.

The operation input section 103 includes various switches, buttons, a track ball, a mouse and a keyboard, for example, for inputting various parameters in order to display a command to start the medical examination, data including personal information of the test object, and the ultrasound image on the display section 106, and outputs operation signals to the control section 107.

The transmission/reception section 104 is a circuit which supplies a driving signal of an electronic signal to the ultrasound probe 102 so as to generate the transmission ultrasound wave, and receives a receiving signal of electronic signal through the ultrasound probe 102, according to the control by the control section 107. The transmission/reception section 104 drives a plurality of oscillators arranged in the ultrasound probe 102 so as to generate the transmission ultrasound wave to perform scanning. The transmission/reception section 104 amplifies the receiving signal with a predetermined amplification factor and conducts an A/D conversion, and then conducts phasing and adding processing, whereby receive beamforming is performed. In other words, it can be said that the transmission/reception section 104 serves as an imaging section which takes an image of an examination region of the test object.

The image processing section 105 performs envelope detecting processing, logarithmic compression, etc. with respect to the receiving signal to which the phasing and adding processing has been conducted, and adjusts a dynamic range and/or a gain to convert a brightness, whereby B-mode image data is created. The B-mode image data represents an intensity of the receiving signal by the brightness. The image processing section 105 creates the ultrasound image data of a frame unit from the B-mode image data created as described above. This ultrasound image data of the frame unit is sometimes called as frame image data. The image processing section 105 includes a high-capacity memory which can hold the frame image data of ten seconds, for example.

The transmission/reception section 104 and the image processing section 105 constitute an image data generation section which generates image data of the examination region of the test object.

As the display section 106, a display device such as an LCD and a CRT display can be adopted. The display section 106 displays the ultrasound image, which is created based on the frame image data created by the image processing section 105, on the display screen.

The control section 107 is configured to include a CPU, ROM and RAM, for example, and reads out the various processing programs such as the system programs stored in the ROM to expand the programs in the RAM, and centrally controls the operations of the respective sections of the ultrasound image diagnosis device 1a according the expanded programs.

The ROM is composed of a nonvolatile memory such as semiconductor, or the like, and stores the system programs with respect to the ultrasound image diagnosis 1a, and various processing programs including a default setting program and an ultrasound examination program, various pieces of data, etc. which are executable on the system programs. Each of the programs is stored in a form of a computer readable program code, and the CPU sequentially executes an operation according to the program code.

The RAM forms a work area for temporarily storing various programs to be executed by the CPU and data regarding these programs.

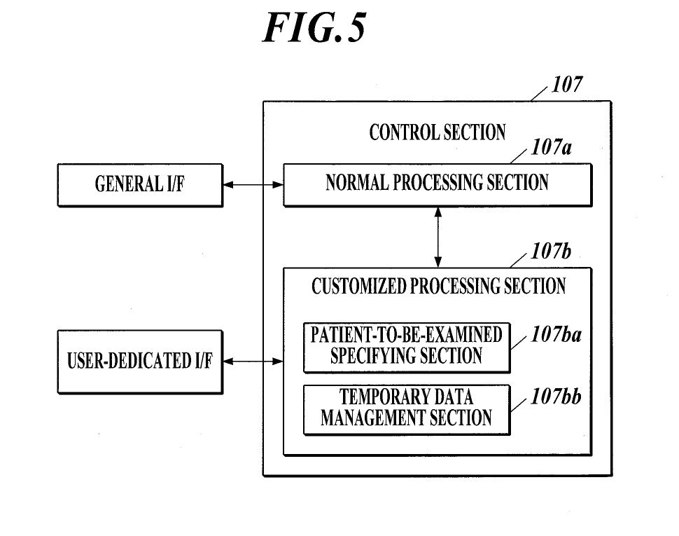

As shown in FIG. 5, the control section 107 includes a normal processing section 107a and a customized processing section 107b.

The normal processing section 107a exerts a general and basic function such as transmitting/receiving processing of the ultrasound wave and processing regarding generation of the ultrasound image data described above. The normal processing section 107a can transmit/receive data to/from outside by using a general I/F.

The customized processing section 107b exerts a user-dedicated function, by which detailed functions can be customized for each user. The customized processing section 107b can transmit/receive data to/from outside by using a user-dedicated I/F. The customized processing section 107b is connected to the normal processing section 107a via the general I/F, for example, and can input/output data to/from each other. According to such configuration, the customized processing section can deal with the case of providing the user-dedicated function without any change in the normal processing section, and customizing can be easily performed.

The control section 107 of the embodiment includes the normal processing section 107a and the customized processing section 107b, but the present invention is not limited to such configuration. The configuration where the normal processing section 107a exerts the function of the customized processing section 107b can be adopted, for example.

The customized processing section 107b includes a patient-to-be-examined specifying section 107ba and a temporary data management section 107bb.

The patient-to-be-examined specifying section 107ba specifies the patient from patient-to-be-displayed identification information as test-object specifying information, which will be described in detail later, transmitted from the electronic medical chart terminal 3.

The temporary data management section 107bb creates temporary patient information as temporary test-object information for temporary management, which will be described in detail later, in the case that the patient-to-be-displayed identification information is not transmitted from the electronic medical chart terminal 3.

In the above configuration, the patient-to-be-examined specifying section 107ba and the temporary data management section 107bb are provided inside the control section 107 of the ultrasound image diagnosis device 1a, but they can be provided outside of the ultrasound image diagnosis device 1a.

The storage section 108 is composed of a high-capacity recording medium such as an HDD, and can store the ultrasound image data created as described above. The storage section 108 can store, as the ultrasound image data, still image data, namely data of still image of one (1) frame, and moving image data which is created from pieces of still image data of plural frames so as to enable a movie display. In the embodiment, also after-mentioned patient information (test-object information) is stored. In other words, the storage section 108 functions as a test-object information storage section in which the test-object information is stored. In addition to the above-mentioned HDD, it is possible to provide a portable recording medium including a Compact Disk-Recordable (CD-R), and a data reading/writing device such as a CD-drive used for recording data in the portable recording medium, which constitute the storage section 108. The storage section 108 can be constituted to be able to store an image file composed of the DICOM image data created as described above.

The communication section 109 is provided with an LAN adaptor, and transmits/receives data between the ultrasound image diagnosis device 1a and the respective devices connected to one another via the LAN 8.

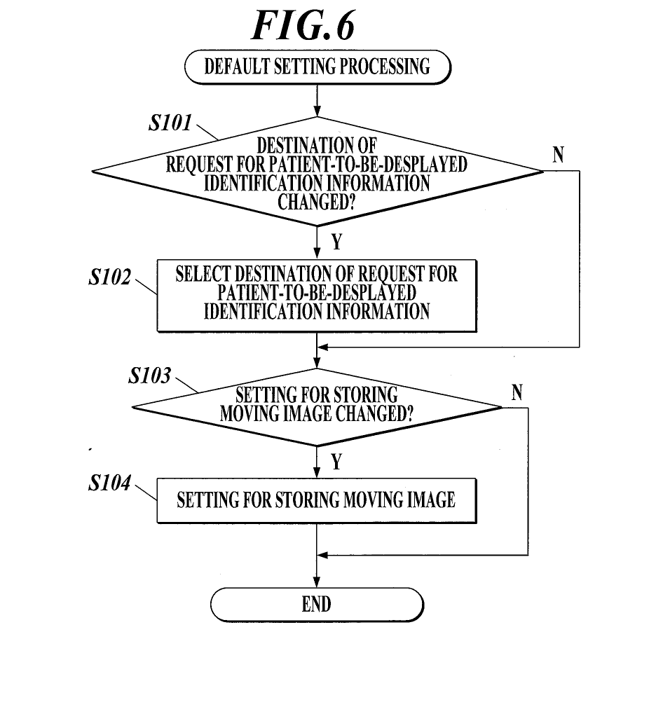

Next, the default setting processing to be executed by the control section 107 of the ultrasound image diagnosis device 1a in the medical image management system 1000 constituted as described above will be described with reference to FIG. 6. The default setting processing is executed according to a predetermined default setting operation by an operator including a doctor and a technician, for example.

The control section 107 firstly determines whether or not the operator performs an operation to change destination of request for patient-to-be-displayed identification information (S101). The destination of request for patient-to-be-displayed identification information can be arbitrary set from among the electronic medical chart terminal 3, the receipt terminal 4, and the PACS terminal 5, depending on a scale of the facilities of hospital/office/clinic and/or the configuration of the medical image management system 1000.

When determining that the operation to change the destination of request for patient-to-be-displayed identification information is performed (Step S101: Y), the control section 107 performs setting of the destination of request for patient-to-be-displayed identification information according to the changing operation by the operator (Step S102). When not determining that the operation to change the destination of request for patient-to-be-displayed identification information is performed (Step S101: N), the control section 107 performs processing of Step S103 without performing the processing of Step S102.

Then, control section 107 determines whether or not an operation to change setting for storing the moving image is performed (Step S103). When determining that the operation to change the setting for storing the moving image is performed (Step S103: Y), the control section 107 performs the setting for storing the moving image (Step S104), and then terminates this processing. According to such setting for storing the moving image, in the case that a compression format of a moving image data file readable by the PACS terminal 5 has been specified, for example, when generating a moving image file from the moving image data in the ultrasound image diagnosis device 1a, it is possible to perform previous setting so that the moving image file is created in the specified compression format. By the setting for storing the moving image, previous setting can also be performed to prevent transmitting the moving image file to the image server 6 when the image server 6 cannot store the moving image file. By the setting for storing the moving image, in the case that a file size of the moving image file readable by the PACS terminal 5 is limited within a predetermined size, previous setting can also be performed to transmit only the moving image file whose size is within the predetermined size to the image server 6. Moreover, by the setting for storing the moving image, it is possible to perform setting for switching a transmission destination depending on the situation when the ultrasound image diagnosis device 1a is connected to a plurality of image servers. Thus, the previous setting can be conducted in the ultrasound image diagnosis device 1a of the embodiment in order to easily and properly store the moving image file correspondingly a reading condition in the PACS terminal 5, a storing condition of the moving image file in the image server 6, etc. The ultrasound image diagnosis device 1a can create the moving image file according to the set contents to transmit the created file to the image server 6 to store the same therein.

On the other hand, when not determining that the operation to change the setting for storing the moving image is performed (Step S103: N), the control section 107 terminates this processing without performing the processing of Step S104.

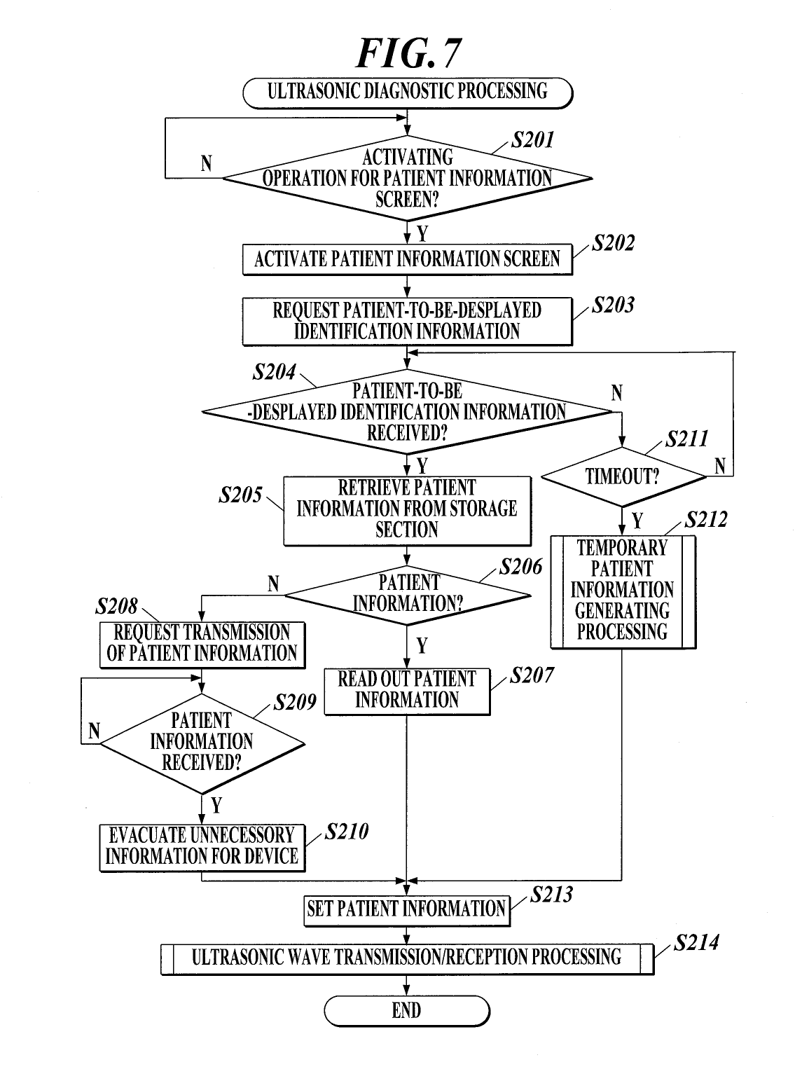

Next, the ultrasound diagnostic processing to be executed by the control section 107 of the ultrasound image diagnosis device 1a will be described with reference to FIG. 7. The ultrasound diagnostic processing is executed after activating the ultrasound image diagnosis device 1a, for example.

In an actual scene of the medical examination, after the doctor conducted the medical examination on the patient, the doctor sometimes uses the ultrasound image diagnosis device in the spot just like using a stethoscope without issuing a so-called examination order. In this case, since the patient currently displayed in the electronic medical chart terminal is the patient to be examined, for example, automatically obtaining the patient information of the patient, which is displayed in the electronic metical chart terminal, in the ultrasound image diagnosis device can save time for inputting the patient information in the ultrasound image diagnosis device, and is very convenient. This embodiment enables improvement in convenience as described below.

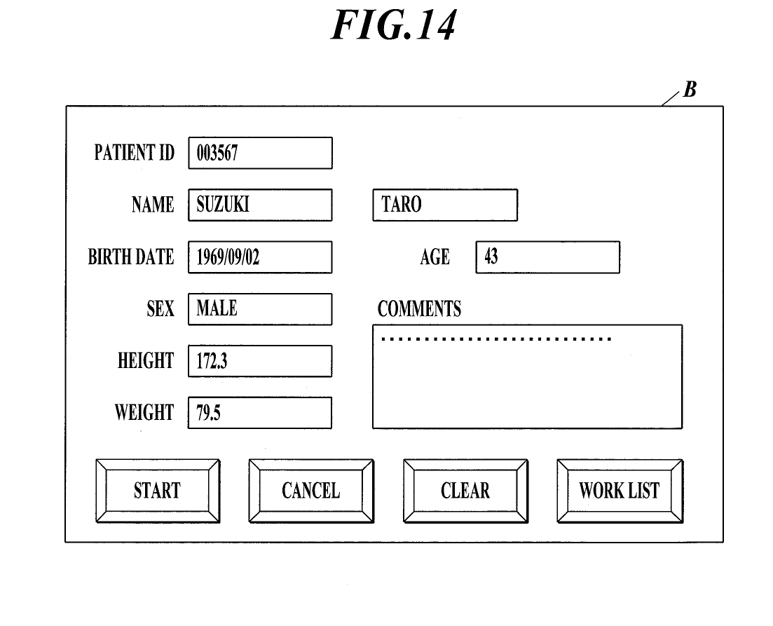

The control section 107 firstly waits until receiving an activating operation for a patient information screen (Step S201), and activates the patient information screen (Step S202). After activating the patient information screen, the control section 107 causes the display section 106 to display a patient information screen B illustrated in FIG. 14, for example. In the patient information screen B, parameters regarding the patient are respectively input in items of the patient ID, name, date of birth, age, sex, height and weight. At the time of activating the patient information screen B, since the information regarding the patient has not been input, the items are in state that the parameters have not been input.

The control section 107 transmits the request for patient-to-be-displayed identification information to the destination of request for patient-to-be-displayed identification information set in the default setting processing (Step S203). Then, the apparatus (for example, the electronic medical chart terminal 3) as the destination of request for patient-to-be-displayed identification information transmits, to the ultrasound image diagnosis device 1a, the patient-to-be-displayed identification information by which the patient of the electronic medical chart displayed in the display screen is specified, as described in detail later. The patient-to-be-displayed identification information is the patient ID written in the electronic medical chart, for example, but the information is not limited to that as long as the information can specify the patient. The embodiment sets the destination of request for the patient-to-be-displayed identification information to the electronic medical chart terminal 3, for example, and the request for the patient-to-be-displayed identification information is transmitted to the electronic medical chart terminal 3. In the case that the destination of request for the patient-to-be-displayed identification information is set to the receipt terminal 4 or the PACS terminal 5, the request for the patient-to-be-displayed identification information is transmitted to the receipt terminal 4 or the PACS terminal 5.

The control section 107 determines whether or not the patient-to-be-displayed identification information has been received from the destination of the request for the patient-to-be-displayed identification information (Step S204). When determining that the patient-to-be-displayed identification information has been received (Step S204: Y), the patient-to-be-examined specifying section 107ba exerts a function to retrieve the patient information corresponding to the received patient-to-be-displayed identification information from the storage section 108 (Step S205). The patient information includes the parameters respectively corresponding to the items on the patient information screen B shown in FIG. 14, and is sometimes called as test-object information.

The control section 107 determines whether or not the corresponding patient information is stored in the storage section 108 (Step S206), and when determining that the corresponding patient information is stored in the storage section 108 (Step S206: Y), reads out the patient information from the storage section 108 (Step S207). On the other hand, when not determining the corresponding patient information is stored in the storage section 108 (Step S206: N), requests the medical examination information server 7 to transmit the patient information (Step S208). The request for transmitting the patient information is conducted by transmitting the patient ID regarding the transmission request, for example. The medical examination information server 7 then retrieves the patient information requested by the ultrasound image diagnosis device 1a by using the patient ID as a retrieval key, as described later, for example, and reads out the corresponding patient information to transmit the same to the ultrasound image diagnosis device 1a.

When receiving the patient information from the medical examination information server 7 (Step S209), the control section 107 extracts information (unnecessary information for the device), which is unnecessary for medical ultrasound examination by using the ultrasound image diagnosis device 1a, from the received pieces of patent information. At that time, the control section 107 determines whether or not a format (for example, a differentiation between the Christian era and the Japanese calendar) of the date included in the patient information transmitted from the medical examination information server 7 agrees with a format of the date managed in the ultrasound image diagnosis device 1a. When the formats of the date agree with each other, the control section 107 uses a parameter of the date included in the patient information transmitted from the medical examination information server 7 as it is. On the other hand, when the formats of the date do not agree with each other, the control section 107 converts the format of the date contained in the patient information transmitted from the medical examination information server 7 into the format adaptable for the ultrasound image diagnosis device 1a, and uses the parameter of the date obtained by the conversion.

The control section 107 temporarily evacuates the unnecessary information for the device, which information has been extracted as described above, to the RAM (Step S210). The unnecessary information for the device is not used in the medical ultrasound examination in the ultrasound image diagnosis device 1a, but when the medical ultrasound examination is terminated and the image file is created, attaching the unnecessary information to the image file contributes to the usability of image file retrieval. The unnecessary information for the device is information which is not input in the ultrasound image diagnosis device 1a, and includes a name of examination place and a name of requested doctor, for example.

When not determining that the patient-to-be-displayed identification information has been received in Step S204 (Step S204: N), the control section 107 determines whether or not a predetermined time has passed after transmitting the request for patient-to-be-displayed identification information and timeout is detected (Step S211). When not determining the timeout is detected (Step S211: N), the control section 107 performs the processing of Step S204. On the other hand, when determining that the timeout is detected (Step S211: Y), the control section 107 makes the temporary data management section 107bb to exert a function to perform temporary patient information generating processing (Step S212). The case that the patient-to-be-displayed identification information is not received includes, for example, the case where the electronic medical chart terminal 3 is not used and the doctor writes remarks in a paper medical chart. Even in such case, the embodiment can omit complicated works such as inputting the patient information when conducting the medical ultrasound examination, in the following way.

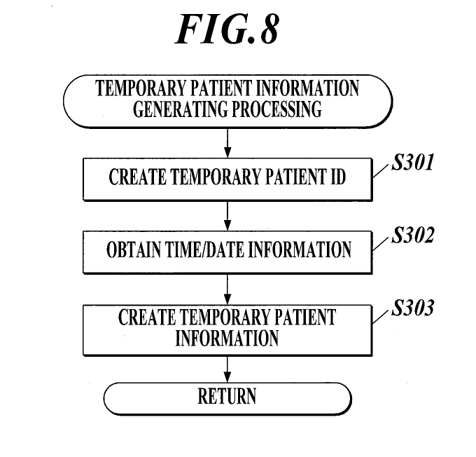

The temporary patient information generating processing will be described with reference to FIG. 8.

The control section 107 firstly creates a temporary patient ID (Step S301). The temporary patient ID is identification information for temporarily managing the patient for which the ultrasound image diagnosis has been conducted. As the temporary patient ID, unique identification number, date, name etc. can be adopted.

Then, the control section 107 obtains time/date information for specifying current time and date from a built-in timepiece section (Step S302). The control section 107 then correlates the obtained time/date information to the temporary patient ID to create temporary patient information (Step S303), and terminates this processing.

As shown in FIG. 7, the control section 107 performs setting of the patient information on the basis of the patient information and/or the temporary patient information read out and/or created as described above (Step S213). By this, the parameters respectively corresponding to the items in the patient information screen B shown in FIG. 14 are input. In the case that the patient information is set on the basis of the temporary patient information, only the patient ID is input. Thus the control section 107 completes the setting of the patient information, then conducts ultrasound wave transmitting/receiving processing (Step S214), and terminates this processing.

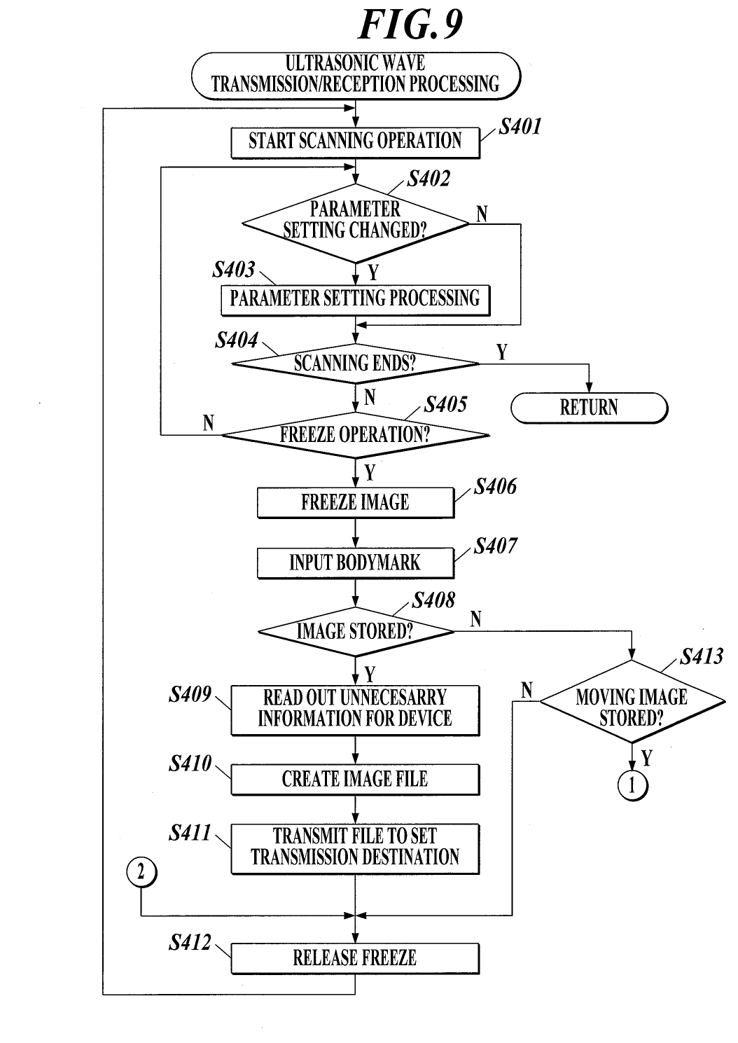

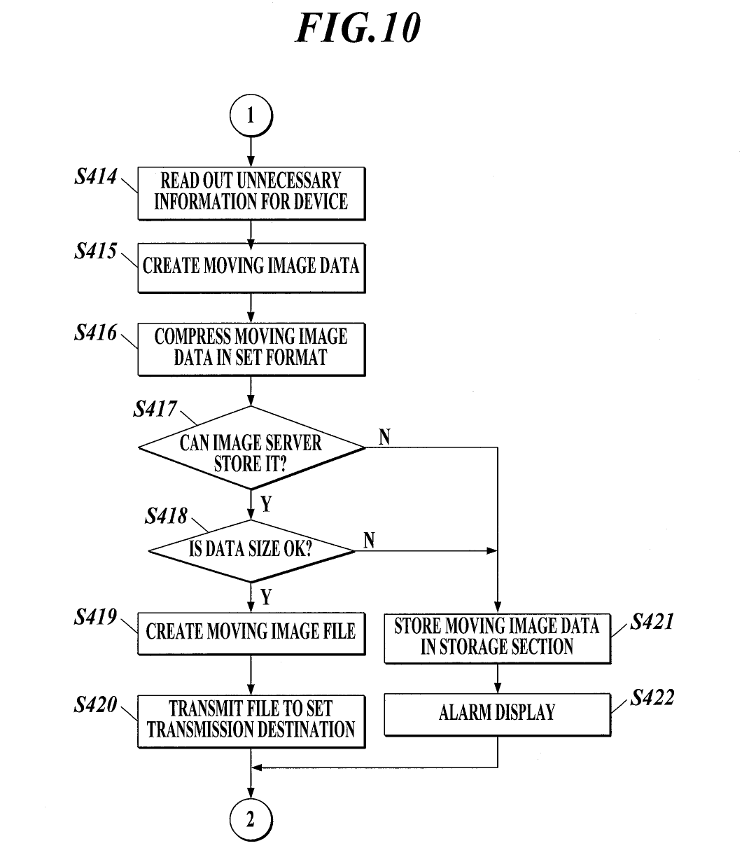

Next, the ultrasound wave transmitting/receiving processing will be described with reference to FIG. 9 and FIG. 10.

The control section 107 firstly starts a scanning operation (Step S401). Concretely, the control section 107 performs transmission/reception of the ultrasound wave though the transmitting/receiving section 104, and makes the image processing section 105 create the ultrasound image data. Then, the control section 107 makes the display section 106 display the ultrasound image based on the created ultrasound image data. By this, real-time display of the ultrasound image is performed in the display section 106. The control section 107 repeatedly performs the above-described operation concurrently also in the following processing.

After that, the control section 107 determines whether or not setting change of various parameters has been performed by inputting in the operation input section 103 (Step S402).

When determining that the setting change of the parameters has been performed (Step S402: Y), the control section 107 conducts setting change of the parameters correspondingly to inputting in the operation input section 103 (Step S403). The parameters which can be set are parameters for displaying the ultrasound image in the display section 106, and include a display depth indicating a focus point, gamma characteristics, gain, dynamic range, etc., for example. On the other hand, when not determining that the setting change of the parameters has been performed (Step S402: N), the control section 107 conducts the processing of Step S404 without performing the processing of Step S403.

The control section 107 determines whether or not the scanning operation is terminated (Step S404). Concretely, the control section 107 determines whether or not receiving a terminating operation in the operation input section 103, which operation is for ending one (1) examination.

When not determining that the scanning operation is terminated (Step S404: N), the control section 107 determines whether or not there has been a freeze operation in the operation input section 103 (Step S405). In other words, the control section 107 determines whether or not an operation to switch the ultrasound image displayed as the moving image in the display section 106 to the still image has been conducted.

When determining that there has been the freeze operation (Step S405: Y), the control section 107 performs an image freezing control for fixedly displaying the ultrasound image which is displayed in the display section 106 at the time of receiving the freeze operation (Step S406). The embodiment stops the scanning operation in the image freeze control, but it is also possible to perform the scanning operation concurrently while fixedly displaying the ultrasound image on the display screen. On the other hand, when not determining there has been the freeze operation (Step S405: N), the control section 107 conducts the processing of Step S402.

Next, the control section 107 receives inputting for selecting a bodymark from the operation input section 103 (Step S407). The bodymark is superimposed on the ultrasound image and used for visually specifying the examination region. The bodymark can be made so as to specify, in addition to the examination region, a direction of the ultrasound probe at the time of obtaining the ultrasound image data, and a position in which the ultrasound image data has been obtained. When receiving the inputting for selecting the bodymark, the control section 107 displays the image corresponding to the selected bodymark so as to be superimposed on the ultrasound image fixedly displayed.

The control section 107 then determines whether or not there has been an image storing operation in the operation input section 103 (Step S408). Concretely, the control section 107 determines whether or not the operation to create and store the file of the ultrasound image data has been received, which image is fixedly displayed in the display section 106.

When determining that there has been the image storing operation (Step S408: Y), and when the unnecessary information for the device is temporarily evacuated to the RAM, the control section 107 reads out the unnecessary information for the device (Step S409), and then creates the image file on the basis of the ultrasound image data (still image data) corresponding to the ultrasound image fixedly displayed in the display section 106. Specifically, the control section 107 firstly converts the still image data corresponding to the fixedly-displayed ultrasound image into the image data to be stored. The image data after the conversion is a bitmap image, for example. The image data after the conversion can include data compressed by compression format such as Joint Photographic Experts Group format (JPEG). The control section 107 then causes the patient information including the read-out unnecessary information for device to be contained in the accessory information so that the same is attached to the image data which has been converted as described above, and creates the image file composed of the DICOM image data. The accessory information includes, in addition to the patient information, the medical examination information, series information, image identification information, etc. In the case that the patient information is the temporary patient information, the control section 107 makes the temporary patient information included in the accessory information to be attached to the image data to create a temporary image file.

The patient information includes information regarding the patient such as a sex, birth date, address and job, in addition to the patient name and the patient ID.

The medical examination information includes information regarding an examination ID for identifying the medical examination, examination name, receipt number, examination object and examination content, in addition to the examination date, the time of storing the image file, and the display depth. The information on the storing time is created on the basis of the date/time information output from the built-in timepiece section.

The series information includes information regarding a unit (or series) of medical images for each modality, which images are created in each examination, including a series number, procedure/manipulation, photographing portion, photographing direction, and name of photography technician, in addition to a modality name and a type of ultrasound probe used. The modality name is a name of modality to be used, namely, information indicating the ultrasound image diagnosis device, in the embodiment. The information on the type of probe includes a differentiation between a probe of a linear scan type and a probe of a convex scan type, a frequency type, and so on.

The image identification information is information regarding the medical image including a Service Object-Pair class (SOP) instance Unique IDentifier (UID) which identifies the image, the image number, the image size, and so on.

The control section 107 transmits the image file created as described above to the transmission destination set in the default setting processing (Step S411). According to the setting of the embodiment, the image file is transmitted to the image server 6, but the image file can be transmitted to another apparatus/device.

Then, the control section 107 performs freeze release control to switch the display in the display section 106 from the fixed display to the moving display (Step S412), and conducts the processing of Step S401.

On the other hand, when not determining there has been the image storing operation in Step S408 (Step S408: N), the control section 107 determines whether or not there has been an operation for storing the moving image in the operation input section 103 (Step S413). Concretely, the control section 107 determines whether or not an operation has been received, which operation is for creating and storing the file of moving image data of ultrasound image during last ten seconds before the freeze operation is conducted.

When determining that there has been the operation for storing the moving image (Step S413: Y), the control section 107 reads out the unnecessary information for device temporarily evacuated to the RAM (Step S414), and then creates moving image data on the basis of each piece of ultrasound image data created during last ten seconds (Step S415), as shown in FIG. 10.

The control section 107 compresses the moving image data created in the compression format previously set in the default setting processing (Step S416). The compression format of the moving image data includes Audio-Video Interleaved format (AVI) and Moving Picture Experts Group 2 (MPEG2), for example.

Next; the control section 107 determines whether or not the moving image data created as described above can be stored in the image server 6 (Step S417). Concretely, the control section 107 determines whether or not storing the moving image data in the image server 6 on the basis of the setting for storing the moving image set in the default setting processing. When determining that the moving image data can be stored in the image server 6 (Step S417: Y), the control section 107 determines whether or not the size of the moving image data is permissible (Step S418). Concretely, the control section 107 determines whether or not the size of the moving image data is within a size corresponding to the file size set in the default setting processing.

When determining that the size of the moving image data is permissible (Step S418: Y), the control section 107 causes the patient information including the read-out unnecessary information for device to be included in the accessory information, adds the same to the moving image data created as described above, and creates the moving image file composed of DICOM image data (Step S419). The control section 107 then transmits the created moving image file to the transmission destination set in the default setting professing (Step S420), and conducts the processing of Step S412. The embodiment transmits the moving image file to the image server 6, but the moving image file can be transmitted to another apparatus/device.

When not determining that the moving image data can be stored in the image server 6 in Step S417 (S417: N), or when not determining that the size of the moving image data is permissible in Step S418 (Step S418: N), the control section 107 stores the moving image data in the storage section 108 (Step S421), makes the display section 106 display an alarm display which informs that the moving image file has not been able to be transmitted to the set transmission destination (Step S422), and terminates the processing of Step S412.

After the image file and the moving image file are created as described above, when determining that the scanning operation is to be terminated in Step S404 (Step S404: Y), the control section 107 terminates this processing.

The embodiment performs setting of the patient information before conducting the ultrasound wave transmitting/receiving processing, but the patient information can be set after the ultrasound wave transmitting/receiving processing, or at the time of generating the image file or the moving image file.

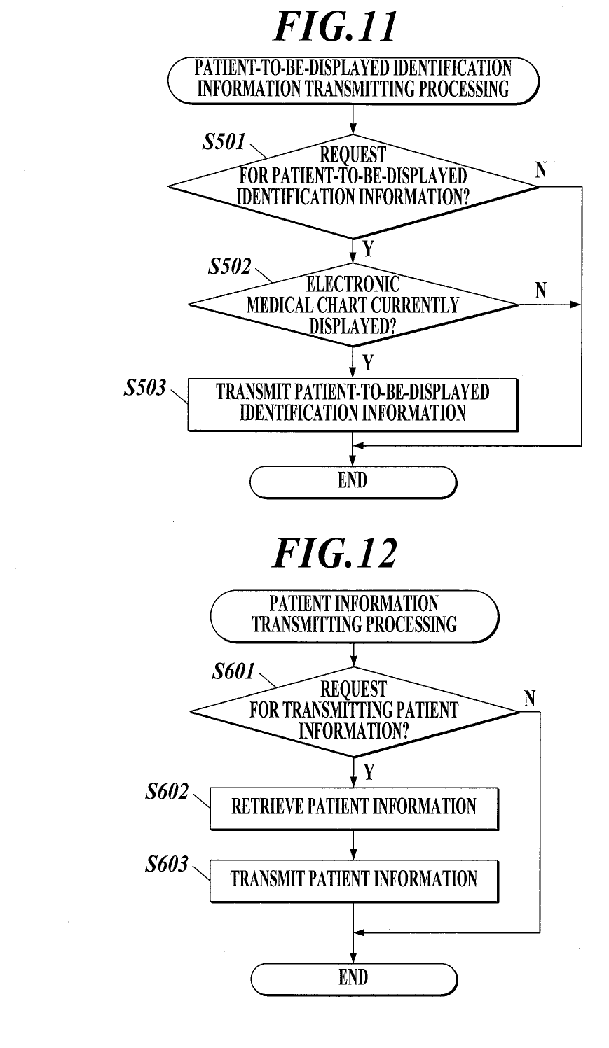

Next, patient-to-be-displayed identification information transmitting processing to be executed by the CPU 31 of the electronic medical chart terminal 3 will be described with reference to FIG. 11.

The CPU 31 firstly determines whether or not there has been the request for patient-to-be-displayed identification information from the medical image generation apparatus 1 such as the ultrasound image diagnosis device 1a and the CR device 1b (Step S501). When determining that there has been the request for patient-to-be-displayed identification information (Step S501: Y), the CPU 31 determines whether or not the electronic medical chart is currently displayed in the display section 35 (Step S502). When determining that the electronic medical chart is currently displayed in the display section 35 (Step S502: Y), the CPU 31 transmits the patient-to-be-displayed identification information corresponding to the patient indicated by the electronic medical chart currently displayed in the display section 35 to the medical image generation apparatus 1 as the transmission source (Step S503), and terminates this processing.

When not determining that there has been the request for patient-to-be-displayed identification information in Step S501 (Step S501: N), or when not determining that the electronic medical chart is currently displayed in the display section 24 (Step S502: N), the CPU 31 terminates this processing without performing the processing of Step S503.

The embodiment describes the case that the present invention is implemented in the electronic medical chart terminal 3, but the present invention can be implemented also in the receipt terminal 4 and/or the PACS terminal 5 by the similar processing. For example, in the case where the doctor writes remarks in the paper medical chart without using the electronic medical chart terminal is adopted, for example, the present invention can be implemented by making the receipt terminal 4 and/or the PACS terminal 5 display the electronic medical chart and/or the information regarding the patient to transmit the patient-to-be-displayed identification information corresponding to the displayed patient.

Next, patient information transmitting processing to be executed in the medical examination information server 7 will be described with reference to FIG. 12. The patient information transmitting processing is performed by the control section 71 of the medical examination information server 7, for example.

The control section 71 firstly determines whether or not there has been the request for transmitting the patient information from the medical image generation apparatus 1 such as the ultrasound image diagnosis device 1a and the CR device 1b (Step S601). When determining that there has been the request for transmitting the patient information (Step S601: Y), the control section 71 retrieves the patient information from the database of the storage section 73, for which information transmission request has been received (Step S602). The control section 71 transmits the patient information extracted as a result of the retrieval to the medical image generation apparatus 1 as the transmission source (Step S603), and terminates this processing.

When not determining that there has been the request for transmitting the patient information in Step S601 (Step S601: N), the control section 71 terminates this processing without performing the processing of the Step S602 or the Step S603.

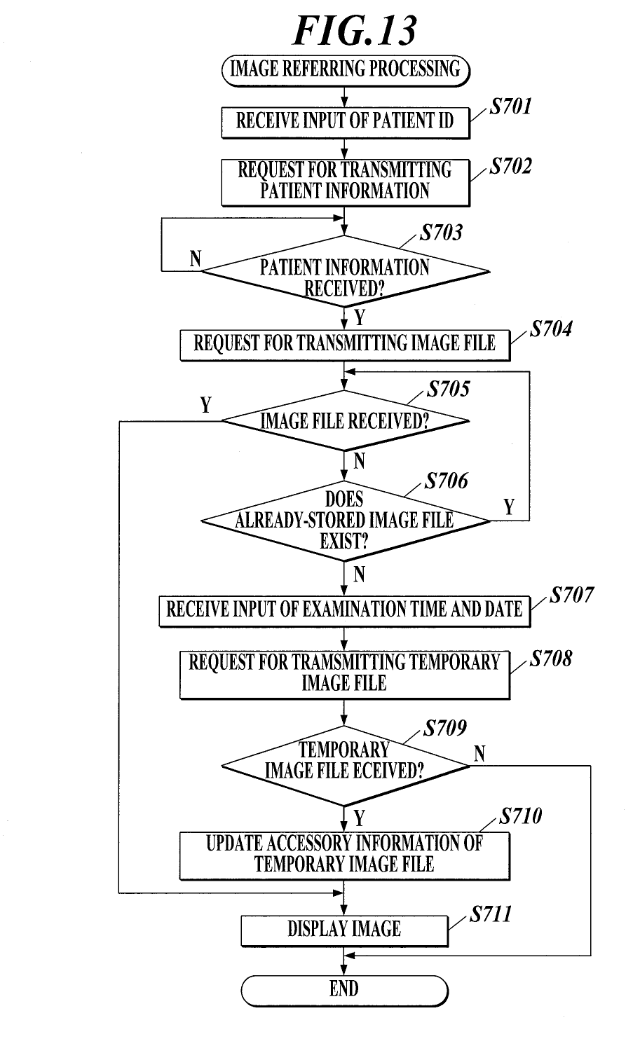

Next, image referring processing to be executed by the PACS terminal 5 will be described with reference to FIG. 13. The image referring processing is executed for displaying the medical image in the display section 55 of the PACS terminal 5 on the basis of the image file stored in the image server 6. The image referring processing is executed by the CPU 51 included in the PACS terminal 5, for example.

The CPU 51 receives an input of the patient ID in the input section 54 including a keyboard etc. (Step S701). The CPU 51 requests the medical examination information server 7 to transmit the patient information corresponding to the patient ID received (Step S702). The CPU 51 waits for receiving the patient information from the medical examination information server 7 (Step S703), and then requests the image server 6 to transmit the image file corresponding to the patient ID (Step S704).

The CPU 51 determines whether or not the image file corresponding to the patient ID has been received from the image server 6 (Step S705). When not determining that the image file corresponding to the patient ID has been received (Step S705: N), the CPU 51 determines whether or not the already-stored image file exists in the image server 6 (Step S706). In other words, the CPU 51 determines whether or not the image file corresponding to the patient ID exists in the image server 6. The judgment whether or not the image file corresponding to the patient ID exists in the image server 6 can be executed by receiving a notice of whether or not the image file corresponding to the patient ID exists in the image server 6 therefrom.

When determining that the already-stored image file exists in the image server 6 (Step S706: Y), the CPU 51 executes the processing of Step S705. On the other hand, when not determining that the already-stored image file exists in the image server 6 (Step S706: N), the CPU 51 receives an input of an examination time and date from the input section 54 (Step S707). The CPU 51 requests the image server 6 to transmit the temporary image file to which the received examination time and date are related (Step S708). The embodiment uses the examination time and date as a retrieval key for temporary image file, but another retrieval key can be used.

Then, the CPU 51 determines whether or not the temporary image file has been received (Step S709). When determining that the temporary image file has been received (Step S709: Y), the CPU 51 updates the accessory information attached to the temporary image file to the patient information received from the medical examination information server 7 (Step S710). When there is a plurality of temporary image files as subjects of the transmission request, a list of temporary image files as subject candidates can be transmitted from the image server 6 to the PACS terminal 5 so that the list is displayed in the display section 55, among which candidates subjects of the transmission request can be selected by inputting in the input section 54.

When determining that the image file corresponding to the patient ID has been received in Step S705 (Step S705: Y), the CPU 51 performs the processing of Step S711 without conducting the processing of Steps S706 to S710.

In Step S711, the CPU 51 makes the display section 55 display the medical image on the basis of the image data contained in the image file received from the image server 6 (Step S711), and terminates this processing.

When not determining that the temporary image file has been received in Step S709 (Step S709: N), the CPU 51 terminates this processing without performing processing of Steps S710 to S711.

According to the abovementioned way of the embodiment, the temporary image file which has not been correlated to the patient information can be correlated to the patient information so as to be easily referred to.

Moreover, according to the embodiment, even in the case that the patient to be examined cannot be specified at the time of conducting the ultrasound diagnosis, the examination can be smoothly performed because temporary image file can be created, and the temporary image file can be correlated to the patient information after the ultrasound diagnosis is terminated. As a result, troublesome inputting of the patient information become unnecessary in the ultrasound diagnosis, and the examination can be smoothly performed.

As described above, it can be said that the embodiment includes the following configuration.

Specifically, the medical image management system of the embodiment includes: an information processing apparatus which receives inputting and performs displaying of the information regarding the test object; a medical image generation apparatus which contains an imaging section to take an image of a portion of the test object to be examined and an image processing section to create image data on the basis of the portion to be examined imaged by the imaging section, and which is communicably connected to the information processing apparatus; and a test-object information storage section which stores test-object information. The medical image generation apparatus includes a control section to request the information processing apparatus to transmit test-object specifying information for specifying the test-object, and when not receiving the test-object specifying information from the information processing apparatus, to create temporary test-object information to correlate the created temporary test-object information to the image data created by the image processing section to store the same in a predetermined storage region. The medical image management system includes an image data updating section to read out one piece of the test-object information from the test-object information storage section and read out the image data stored in the predetermined storage region, and to update the content of the temporary test-object information of the image data read out, to the test-object information read out from the test-object information storage section.

In the medical image management system of the embodiment, the control section of the medical image generation apparatus receives the input of the retrieval information to correlate the same to the image data along with the temporary test-object information when generating the temporary test-object information, and the image date updating section receives the input of the retrieval information and reads out the image data corresponding to the received retrieval information from the predetermined storage region.

In the embodiment, the PACS terminal 5 displays the medical image, but another apparatus/device such as the electronic medical chart terminal 3 and the receipt terminal 4 can display the medical image. Moreover, the electronic medical chart terminal 3 can be provided with the function of the PACS terminal 5.

The PACS terminal 5 of the embodiment correlates the patient information to the temporary image file, but such correlating can be performed by another terminal such as the electronic medical chart terminal 3 and the receipt terminal 4.

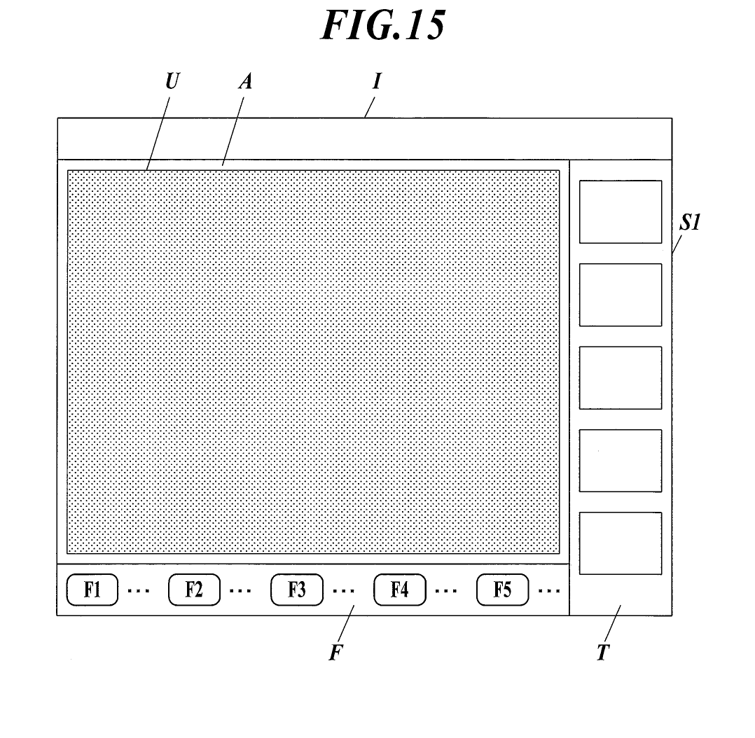

Next, a configuration of the display screen to be displayed in the display section 106 of the ultrasound image diagnosis device 1a of the embodiment will be described with reference to FIG. 15.

As shown in FIG. 15, an image display region A for displaying an ultrasound image U is provided in the center of a display screen S1 displayed in the display section 106. In an upper end portion of the display screen S1, there is provided a basic information display region I in which the personal information of the patient is displayed. In a right end portion of the display screen S1, there is provided a thumbnail display region T in which the still image data and the moving image data already-created in the abovementioned way are displayed in a list. Concretely, pieces of thumbnail data are created correspondingly to the already-created image data and moving image data, and thumbnail images based on the thumbnail data are displayed to be aligned with one another in the thumbnail display region T. In the case that all of the thumbnail images cannot be displayed in one (1) screen, all of the thumbnail images can be displayed by being scroll-displayed, for example. In a lower end portion of the display screen S1, there is provided a key function display region F in which functional explanations of soft keys and/of functional keys are displayed. The function of each of the keys displayed in the key function display region F changes depending on the control status thereof, and accordingly the functional explanation thereof is changed.

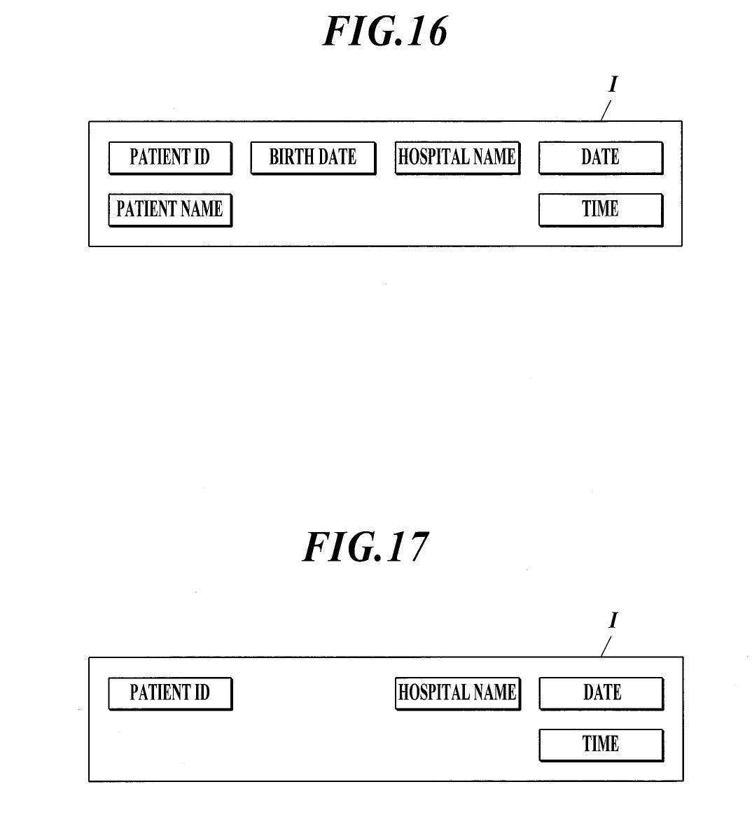

As shown in FIG. 16, the patient ID, patient name, birth date, hospital name, date and time are displayed in the basic information display region I. When creating the still image data and the moving image data, the contents displayed in the basic information display region I are made contained therein.

The embodiment can display all of the contents of the basic information display region I in a normal mode, and additionally, can perform a display in a personal information protection mode by which a part of the contents of the basic information display region I is not displayed in terms of personal information protection and prevent of personal information leak. More concretely, as shown in FIG. 17, the patient name and the birth date are not displayed.

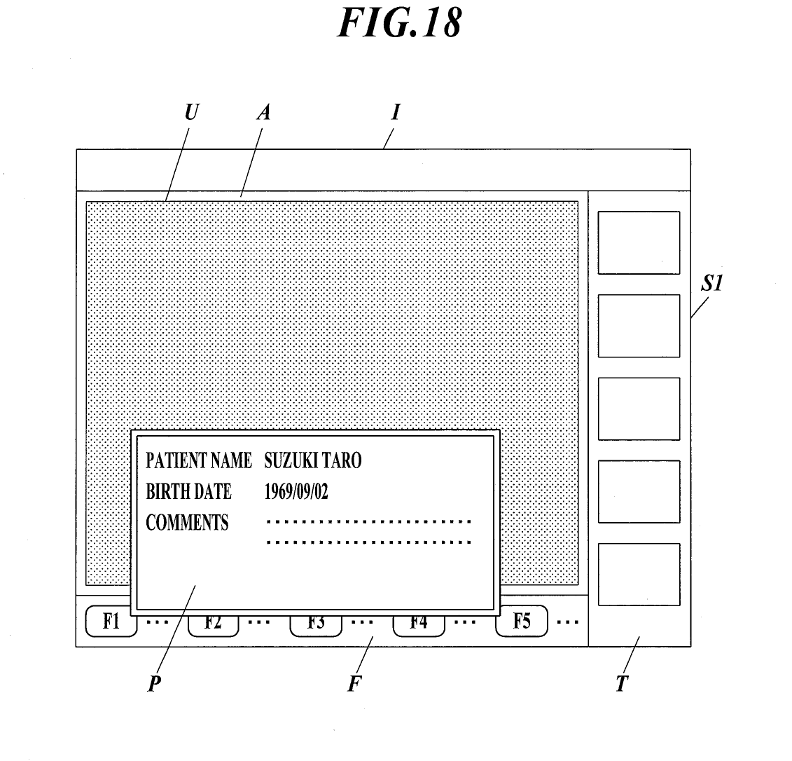

Meanwhile, even when conducting the ultrasound diagnosis in the personal information protection mode, the patient name and the birth date sometimes need to be confirmed. However, since the patient name and the birth date are not displayed in the personal information protection mode, they cannot be confirmed during the ultrasound diagnosis. Even in the case the patient name and the birth date are displayed in the basic information display region I in the normal mode, since the space of the display region is limited, confirming the display is sometimes difficult and misreading can be caused. For this reason, in the embodiment, a pop-up window P can be displayed while superimposing the pop-up window P on the ultrasound image U as shown in FIG. 18 by performing a predetermined operation in the operation input section 103, for example. On the pop-up window P, a part of the patient information such as the patient name and the birth date, detailed comments written at the time of requesting examination/inspection, and the like are displayed. The patient information and the detailed comments displayed on the pop-up window P are larger than those displayed in the basic information display region I, and can be displayed so as to be easily confirmed. This enables the operator such as the doctor conducting the ultrasound diagnosis to easily confirm the part of the patient information and the detailed comments by a simple operation to promptly perform the examination. The pop-up window P can be deleted by a predetermined operation in the operation input section 103. Also the setting can be performed so that the pop-up window P is automatically deleted when a predetermined time has passed since the pop-up window P is displayed. It is also possible to adopt a configuration where items to be displayed on the pop-up window P can be previously set. In the embodiment, not only the patient information read out from the medical examination information server 7 and/or the ultrasound image diagnosis device 1a, but also a part or all of the patient information directly input in the operation input section 103 of the ultrasound image diagnosis device 1a, and a part or all of the patient information obtained from the examination/inspection order transmitted from another device/apparatus can be displayed on the pop-up window P.

Next, a second embodiment of the medical image management system will be described. The same configurations as the first embodiment are designated by the same numerals, and the detailed descriptions thereof are omitted.

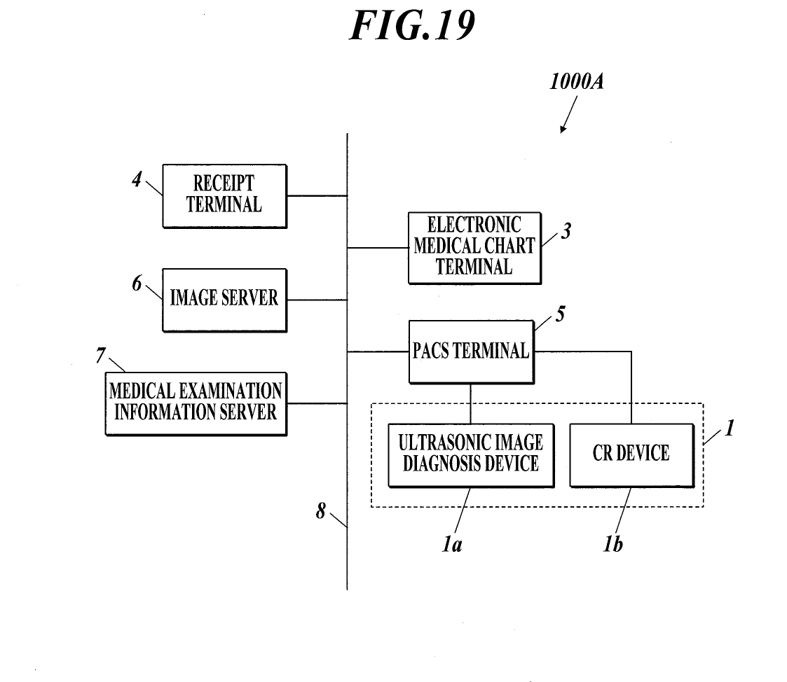

Although the medical image management system 1000 of the first embodiment has the medical image generation apparatus 1 which is directly connected to the LAN 8, the medical image management system 1000A of the second embodiment has the medical image generation apparatus 1 which is connected to the LAN 8 through the PACS terminal 5, as shown in FIG. 19. In other words, the medical image management system 1000A has a configuration where the medical image generation apparatus 1 and the PACS terminal 5 are connected with a local network and the PACS terminal 5 is connected to the electronic medical chart terminal 3 and the image server 6 via the LAN 8.

In the medical image management system 1000A, requests and/or data transmitted from the medical image generation apparatus 1 to the electronic medical chart terminal 3 and/or the image server 6 are transmitted from the medical image generation apparatus 1 through the PACS terminal 5. Like this configuration, the medical image generation apparatus 1 does always not need to be on the same network as other terminals such as PACS terminal 3 as long as the medical image generation apparatus 1 can input/output the data through a mediation apparatus such as the PACS terminal 5.

Next, a third embodiment of the medical image management system will be described. The same configurations as the first embodiment are designated by the same numerals, and the detailed descriptions thereof are omitted.

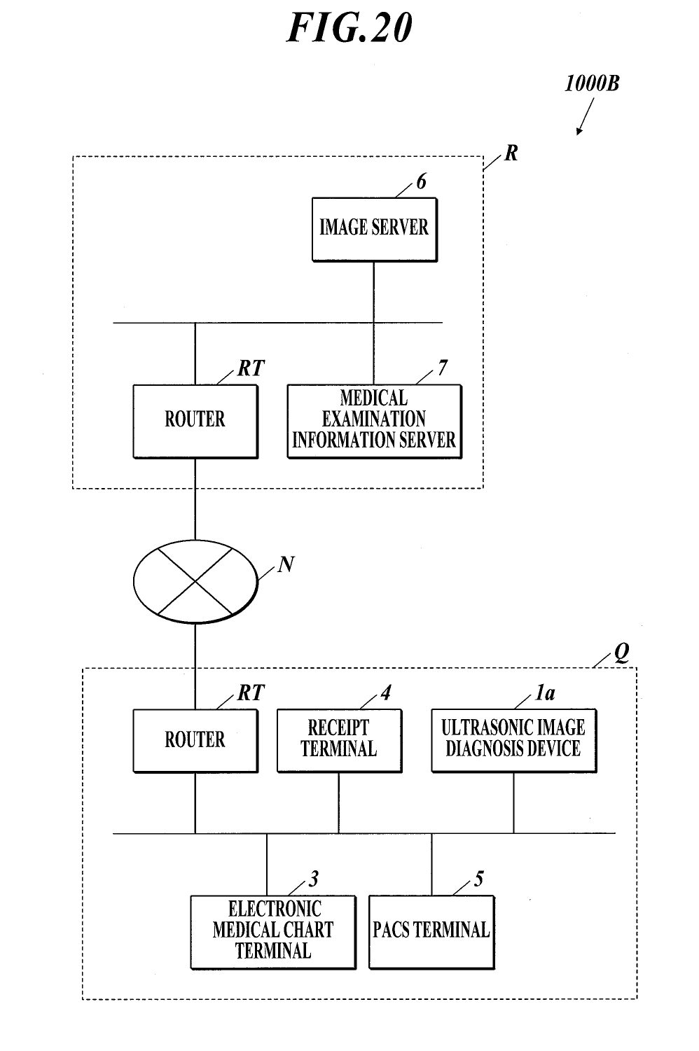

In the medical image management system 1000 of the first embodiment, the medical image generation apparatus 1, the electronic medical chart terminal 3, the receipt terminal 4, the PACS terminal 5, the image server 6, and the medical examination information server 7 are provided in one (1) facility, and they are connected to one another with the LAN 8. In the medical image management system 1000B of the third embodiment, a network including two medical facilities Q, R is formed as shown in FIG. 20. This is a sample configuration of medical network in which a community clinic cooperates with a large hospital, or a sample configuration of data access to a cloud-type server for a medical examination at home. In the medical facility Q, the ultrasound image diagnosis device 1a as the medical image generation apparatus, the electronic medical chart terminal 3, the receipt terminal 4, and the PACS terminal 5 are disposed, and they are connected to one another via the LAN. In the medical facility R, the image server 6 and the medical examination information server 7 are disposed, and they are connected to each other via the LAN. The networks formed in the medical facilities Q and R are connected to a public network N via routers RT. Each of the apparatuses/devices constituting the network can transmit/receive the data via the public network by installing browser software, for example. By this, each of the apparatuses/devices can easily obtain the patient information at low cost from the medical examination information server at remote location by a general public network line and versatile browser software.

Next, another embodiment of the ultrasound image diagnosis device will be described. The same configurations as the above embodiments are designated by the same numerals, and the detailed descriptions thereof are omitted.

Heretofore, there has been known a method for connecting an external inputting apparatus/device to the medical image generation apparatus to specify the patient to be examined to obtain the patient information. As the external inputting apparatus/device, a barcode reader, two-dimensional code reader, magnetic card reader, etc. have been known, for example. When using the magnetic card reader, for example, by reading a magnetic card possessed by the patient to be examined by the magnetic card reader, the medical image generation apparatus can specify the patient to be examined and obtain the patient information.

However, since an interface and/or data format are different depending on a type of the external inputting apparatus, the medical image generation apparatus needs to have a data receiving/processing section corresponding to each of the interface and/or data format.

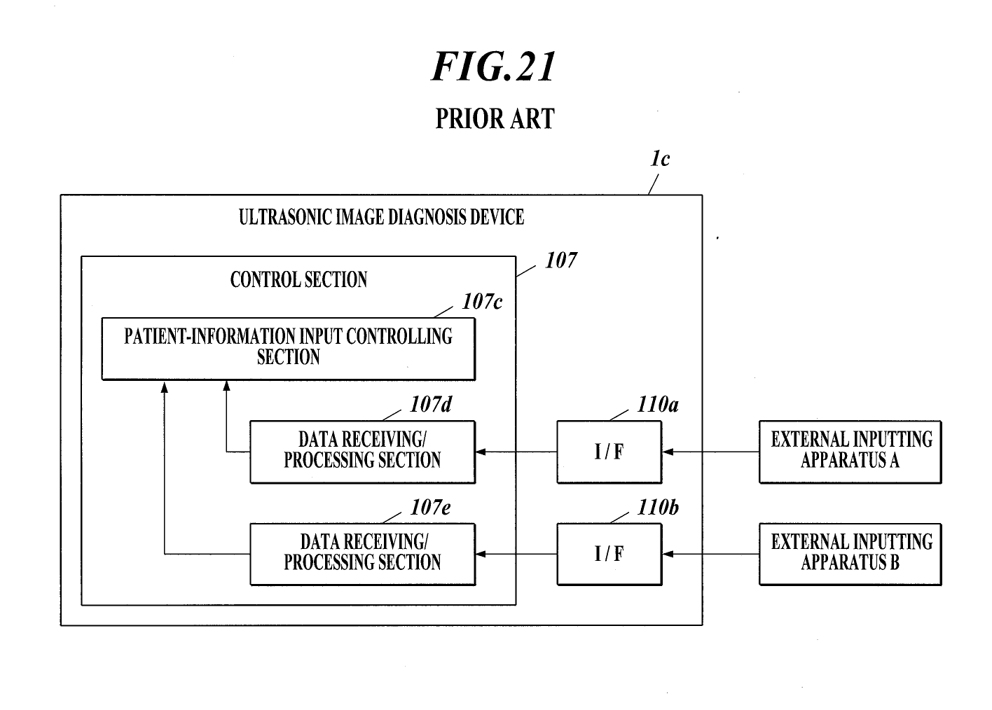

Specifically, as shown in FIG. 21, a control section 107 of a conventional ultrasound image diagnosis device 1c includes a patient-information input controlling section 107c and data receiving/processing sections 107d, 107e. The patient-information input controlling section 107c controls inputting of the patient information input form the data receiving/processing sections 107d, 107e. The data receiving/processing sections 107d, 107e are composed of device drivers, are connected to I/F 110a and I/F 110b respectively, and process the patient information input from the I/F 110a and I/F 110b. The I/F 110a and I/F 110b are connected to external inputting apparatuses A, B respectively, and receive inputs of the patient information transmitted from the external inputting apparatuses A, B.

As described above, the conventional ultrasound image diagnosis device 1c needs to provide the data receiving/processing sections 107d, 107e for respective connections with the external inputting apparatuses. For this reason, the conventional ultrasound image diagnosis device 1c lacks versatility and has complicated configuration.

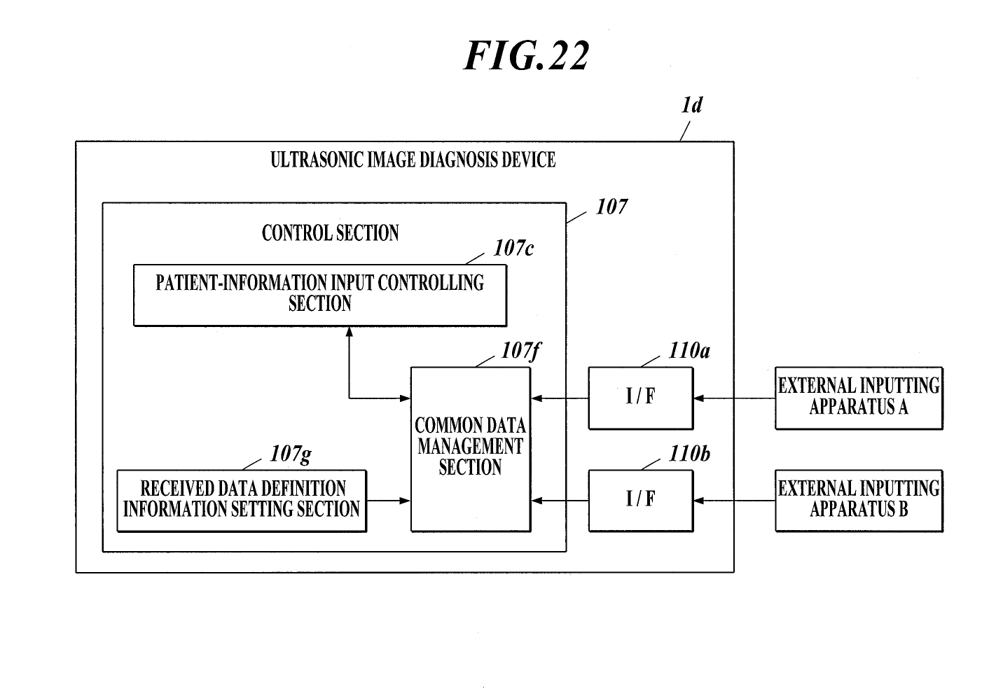

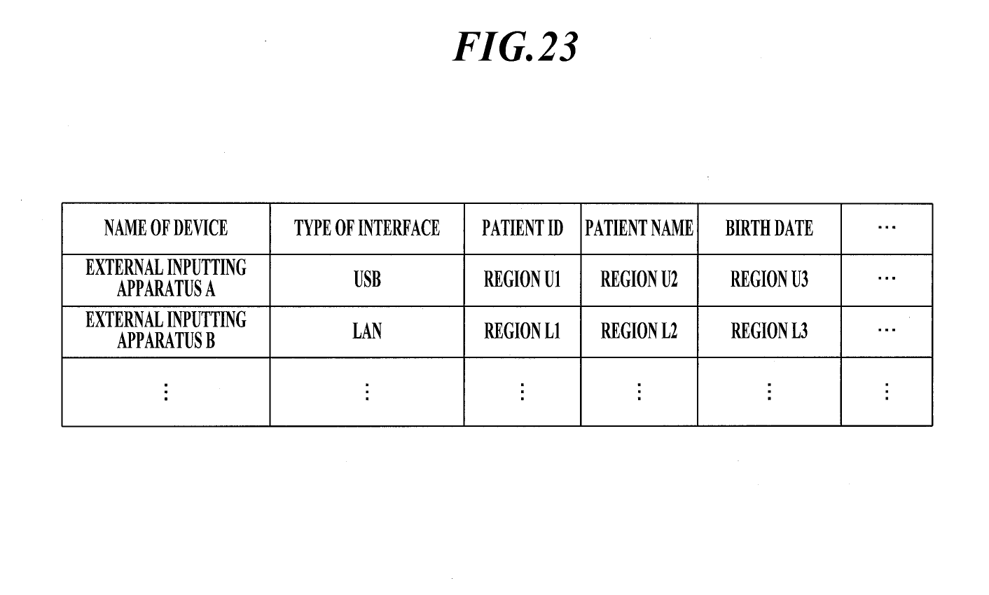

On the other hand, as shown FIG. 22, the ultrasound image diagnosis device 1d according to another embodiment is provided with a common data management section 107f and a received data definition information setting section 107g, instead of the data receiving/processing sections. The received data definition information setting section 107g provides the common data management section 107f with external-inputting-apparatus received-data definition information. The external-inputting-apparatus received-data definition information includes, for example, previously-defined information corresponding to each of the external inputting apparatuses A, B, such as an interface type, and information for defining a data storage region for each item of the patient information, as shown in FIG. 23. The received data definition information setting section 107g gives the external-inputting-apparatus received-data definition information corresponding to the types of the external inputting apparatuses A, B and/or the I/F 110a and I/F 110b connected thereto. By this, the common data management section 107f can recognize the type of the external inputting apparatus connected to the ultrasound image diagnosis device 1d, and the I/F to which the external inputting apparatus is connected, on the basis of the external-inputting-apparatus received-data definition information given from the received data definition information setting section 107g. When the patient information is input from the external inputting apparatuses A, B, the common data management section 107f can refer to the external-inputting-apparatus received-data definition information to interpret the content of the patient information. The common data management section 107f manages the patient information for each of the items. When the control section 107 obtains necessary item information among the pieces of the patient information, the control section 107 makes the patient information input controlling section 107c function to request the necessary item information with respect to the common data management section 107f. When receiving the request for the information from the patient information input controlling section 107c, the common data management section 107f transmits the patient information of the corresponding item to the patient information input controlling section 107c. In the case of obtaining the patient ID information among the pieces of patient information, for example, the control section 107 makes the patient information input controlling section 107c function to request the patient ID information with respect to the common data management section 107f. When receiving the request for the patient ID information from the patient information input controlling section 107c, the common data management section 107f transmits the patient ID information (parameter) included in the patient information. When receiving the patient ID information from the patient information input controlling section 107c, the control section 107 performs processing based on the received patient ID information. Incidentally, in the case that the patient information of the item requested from the patient information input controlling section 107c is not included in the patient information transmitted from the external inputting apparatus, the common data management section 107f transmits information informing that state to the patient-information input controlling section 107c.

By this, the patient-information input controlling section 107c can receive the patient information of necessary item regardless of the type of the external inputting apparatus. According to this configuration, it is not necessary to individually provide the data receiving/processing sections corresponding to the external inputting apparatuses, and the whole configuration of the device can be simplified. Even when the types of the external inputting apparatuses are increased, design change of the control section 107 becomes unnecessary, because the received data definition information setting section 107g can provide the external-inputting-apparatus received-data definition information correspondingly to the increased type of the external inputting apparatuses.

As described above, it can be said that the embodiment includes the following configuration.

Specifically, the ultrasound image diagnosis device of this embodiment includes: interfaces connected to a plurality types of external inputting apparatuses each outputting the patient information regarding the patient; a common data management section which is connected to the interfaces and interprets the patient information from the external inputting apparatuses; and received-data definition information setting section which provides the common data management section with the received-data definition information corresponding to the types of the external inputting apparatuses connected to the interfaces, which information is used for interpreting the patient information input from the external inputting apparatuses.

As described above, according to this embodiment, the transmitting/receiving section 104 takes an image of an examination portion of the test object. The image processing section 105 creates image data on the basis of the examination portion imaged by the transmitting/receiving section 104. The control section 107 requests the electronic medical chart terminal 3 to transmit the patient-to-be-displayed identification information for specifying the test object. The control section 107 receives the patient-to-be-displayed identification information transmitted from the electronic medical chart terminal 3. The control section 107 obtains the patient information corresponding to the received patient-to-be-displayed identification information from the storage section 108 (the medical examination information server 7). The control section 107 correlates the obtained patient information to the image data created by the image processing section 105. According to this, since the patient as the test object can be automatically specified from the apparatus/device outside the medical image generation apparatus in the medical examination using the medical image generation apparatus, setting of the patient information can be surely performed, and easily conducted by simple operations without specific facilities.

The control section 107 of the embodiment requests the electronic medical chart terminal 3 to transmit the patient-to-be-displayed identification information depending on a predetermined operation in the operation input section 103. According to this, since the patient as the test object can be specified at arbitrary timing for the operator, processing load of the apparatus/device and/or the communication load on the network can be suppressed.

The control section 107 of the embodiment also requests the electronic medical chart terminal 3 to transmit the patient-to-be-displayed identification information, which terminal 3 creates medical examination record data of the test object, and receives the patient-to-be-displayed identification information from the electronic medical chart terminal 3. According to this, the operator does not need to conduct an operation to specify the patient as the test object in the medical image generation apparatus, and the patient information of the patient as the test object can be automatically obtained. Especially, the embodiment is very convenient in a scene where a practitioner, who uses the electronic medical chart in his office, uses the medical image generation apparatus such as the ultrasound image diagnosis device just like using a stethoscope.

Moreover, the control section 107 of the embodiment requests the receipt terminal 4 which creates the receipt information to transmit the patient-to-be-displayed identification information, and receives the patient-to-be-displayed identification information from the receipt terminal 4. According to this, the patient information of the patient as the test object can be automatically obtained without an operation by the operator for specifying the patient as the test object in the medical image generation apparatus. Especially, in the case that the use of the electronic medical chart with the electronic medical chart terminal is not adopted and the receipt terminal manages the patient as the test object, the patient information of the patient as the test object can be automatically input.

Furthermore, the control section 107 of the embodiment requests the PACS terminal 5 managing the image data to which the accessory information is attached to transmit the patient-to-be-displayed information, and receives the patient-to-be-displayed information from the PACS terminal 5. According to this, the patient information of the patient as the test object can be automatically obtained without an operation by the operator for specifying the patient as the test object in the medical image generation apparatus. The embodiment is very convenient especially in the case that the use of the electronic medical chart with the electronic medical chart terminal is not adopted and the PACS terminal is used.

The transmitting/receiving section 104 of the embodiment outputs the transmission ultrasound wave to the test object, and receives the reflective ultrasound wave from the test object to obtain the receiving signal to take the image of the examination region of the test object. The image processing section 105 creates the ultrasound image data on the basis of the receiving signal obtained from the transmitting/receiving section 104. According to this, when using the ultrasound image diagnosis device, a step for inputting the patient information can be omitted and the ultrasound image diagnosis device can be easily used just like using a stethoscope, which improves usability.

The electronic medical chart terminal 3 of the embodiment receives inputting and performs displaying of the information regarding the test object. The ultrasound image diagnosis device 1a includes the transmitting/receiving section 104 which takes the image of the examination region of the test object, and the image processing section 105 which creates the image data on the basis of the examination region imaged by the transmitting/receiving section 104, and is communicably connected to the electric medical chart terminal 3. The storage section 108 (the medical examination information server 7) stores the patient information. The control section 107 requests the electronic medical chart terminal 3 to transmit the patient-to-be-displayed identification information for specifying the test object. The control section 107 receives the patient-to-be-displayed identification information transmitted from the electronic medical chart terminal 3. The control section 107 obtains the patient information corresponding to the received patient-to-be-displayed identification information from the storage section 108 (the medical examination information server 7). The control section 107 correlates the obtained patient information to the image data created by the image processing section 105. In the case of receiving the request for transmitting the patient-to-be-displayed identification information from the ultrasound image diagnosis device 1a, the CPU 31 specifies the information regarding one (1) test object for which the information is input and displayed in the electronic medical chart terminal 3. The CPU 31 transmits the identification information corresponding to the information regarding the specified test object as the patient-to-be-displayed identification information to the medical image generation apparatus 1. According to this, since the patient as the test object can be automatically specified from the apparatus/device outside the medical image generation apparatus in the medical examination using the medical image generation apparatus, setting of the patient information can be surely performed, and easily conducted by simple operations.

Moreover, the control section 107 of the embodiment performs setting about which of the plurality of information processing apparatuses receives the request for transmitting the patient-to-be-displayed identification information. According to this, it becomes possible to specify most appropriate apparatus/device for specifying the patient as the test object depending on the situation even when the plurality of information processing apparatuses is connected thereto, and thereby the patient as the test object can be appropriately specified.

Furthermore, the control section 107 of the embodiment creates the temporary patient ID when the patient-to-be-displayed identification information is not received from the electronic medical chart terminal 3, and correlates the created temporary patient ID to the image data created by the image processing section 105 to store the same in the predetermined storage region. The CPU 51 of the PACS terminal 5 reads out one (1) piece of the patient information from the medical examination information server 7, reads out the image data stored in the predetermined storage region, and updates the contents of the temporary patient ID of the image data read out to the patient information read out from the medical examination information server 7. According to this, even when the patient as the test object cannot be automatically obtained in the apparatus/device outside of the medical image generation apparatus in the medical examination using the medical image generation apparatus, specification can be executed by simple operations. After the medical examination using the medical image generation apparatus is terminated, for example, the image data can be correlated to the original patient information. By this, correlation can be conducted without complicated operations in the medical examination using the medical image generation apparatus, and the image data can be properly managed.

Incidentally, the descriptions in the embodiments of the present inventions are mere examples of the medical image management system of the present invention, and the present invention is not limited to the embodiments. The detailed configurations and operations of the functional sections constituting the medical image management system can be arbitrarily changed.

In the embodiment, it is also possible to receive an examination/inspection order from the electronic medical chart terminal 3 to perform the ultrasound diagnosis in the ultrasound image diagnosis device 1a on the basis of the order. In this case, the examination/inspection order transmitted from the electronic medical chart terminal 3 sometimes includes information unnecessary for the ultrasound diagnosis by the ultrasound image diagnosis device 1a such as a name of examination place and a name of requested doctor. These pieces of information is unnecessary for the ultrasound diagnosis by the ultrasound image diagnosis device 1a, but sometimes necessary for retrieving the image file in the PACS terminal 5 or the like and including these pieces of information in the image file is sometimes convenient. In the embodiment, among the pieces of information contained in the examination/inspection order, the information unnecessary for the ultrasound diagnosis by the ultrasound image diagnosis device 1a can be temporarily evacuated to the RAM of the control section 107 so as to be read out when creating the image file and attached thereto, for example, while the information used in the ultrasound image diagnosis device 1a is used as the parameters of the respective items of the patient information screen B shown in FIG. 14B.

Although in the embodiment the electronic medical chart terminal or the like transmits the identification information such as the patient ID as the patient-to-be-displayed information to the medical image generation apparatus, the electronic medical chart terminal or the like can obtain a hard copy of the electronic medical chart displayed on the display screen to transmit the hard copy to the medical image generation apparatus so that the medical image generation apparatus analyzes the content of the hard copy by using a function such as Optical Character Reader (OCR) to extract the patient-to-be-displayed identification information.

Although in the embodiment the unnecessary information for the device is included in the accessory information attached to the image file and/or the moving image file, the unnecessary information does always not need to be included.

Moreover, though in the embodiment the ultrasound image diagnosis device performs the request for transmitting the patient-to-be-displayed identification information, such configuration can be applied to the CR device or another modality.

Furthermore, though the embodiment discloses the example where the hard disk, the nonvolatile semiconductor memory, or the like is used as the computer readable medium for the program of the present invention, the present invention is not limited to the example. As another computer readable medium, a portable recording medium such as CD-ROM can be adopted. In addition, as a medium for providing data of the program of the present invention via a communication line, a carrier wave can be adopted.

The present U.S. patent application claims a priority under the Paris Convention of Japanese patent application No. 2012-160091 filed on Jul. 19, 2012, in which all contents of this application are disclosed, and which shall be a basis of correction of an incorrect translation.

What is claimed is:

1. A medical image generation apparatus communicably connected to an information processing apparatus which receives inputting and performs displaying of information regarding a test object, the medical image generation apparatus comprising:
an image data generation section to generate image data of the examination region of the test object; and
a control section to request the information processing apparatus to transmit test-object specifying information for specifying the test object, to receive the test-object specifying information transmitted from the information processing apparatus, to obtain test-object information corresponding to the received test-object specifying information from a predetermined test-object information storage section, and to correlate the obtained test-object information to the image data created by the image data generation section.

2. The medical image generation apparatus of claim 1 further comprising:
an operation section, wherein
the control section requests the information processing apparatus to transmit the test-object specifying information depending on a predetermined operation in the operation section.

3. The medical image generation apparatus of claim 1, wherein
the control section requests an electronic medical chart terminal as the information processing apparatus to transmit the test-object specifying information, the electronic medical chart terminal being used to create medical record data of the test object, and receives the test-object specifying information from the electronic medical chart terminal.

4. The medical image generation apparatus of claim 1, wherein
the control section requests a receipt terminal as the information processing apparatus to transmit the test-object specifying information, the receipt terminal being used to create receipt information, and receives the test-object specifying information from the receipt terminal.

5. The medical image generation apparatus of claim 1, wherein
the control section requests a medical image management apparatus as the information processing apparatus to transmit the test-object specifying information, the medical image management apparatus being used to manage the image data to which accessory information is attached, and receives the test-object specifying information from the medical image management apparatus.

6. The medical image generation apparatus of claim 1, wherein
the image data generation section outputs transmission ultrasound wave toward the test object, receives reflective ultrasound wave from the test object to obtain a receiving signal, and creates ultrasound image data on the basis of the obtained receiving signal.

7. A medical image management system comprising:
at least one (1) information processing apparatus which receives inputting and performs displaying of information regarding a test object;
a medical image generation apparatus which includes an image data generation section to generate image data of the examination region of the test object, and which is communicably connected to the information processing apparatus; and
a test-object information storage section to store test-object information, wherein
the medical image generation apparatus includes a control section which requests the information processing apparatus to transmit test-object specifying information for specifying the test object, receives the test-object specifying information transmitted from the information processing apparatus, obtains the test-object information corresponding to the received test-object specifying information from the test-object information storage section, and correlates the obtained test-object information to the image data created by the image data generation section, and
the information processing apparatus includes an information transmission controlling section which specifies information regarding one (1) test object for which information is input and displayed in the information processing apparatus, and transmits identification information corresponding to the specified information regarding the test object as the test-object specifying information to the medical image generation apparatus, when receiving a request for transmitting the test-object specifying information from the medical image generation apparatus.

8. The medical image management system of claim 7, wherein
the medical image generation apparatus further includes an operation section, and
the control section of the medical image generation apparatus requests the information processing apparatus to transmit the test-object specifying information depending on a predetermined operation in the operation section.

9. The medical image management system of claim 7, wherein
the at least one information processing apparatus is a plurality of information processing apparatuses, each of which is communicably connected to the medical image generation apparatus, and
the control section of the medical image generation apparatus performs setting about which of the information processing apparatuses is requested to transmit the test-object specifying information.

10. The medical image management system of claim 7, wherein
the information processing apparatus includes an electronic medical chart terminal used to create medical record data of the test object, and
the control section of the medical image generation apparatus requests the electronic medical chart terminal to transmit the test-object specifying information.

11. The medical image management system of claim 7, wherein
the information processing apparatus includes a receipt terminal which creates receipt information, and
the control section of the medical image generation apparatus requests the receipt terminal to transmit the test-object specifying information.

12. The medical image management system of claim 7, wherein
the information processing apparatus includes a medical image management apparatus which manages the image data to which accessory information is attached, and
the control section of the medical image generation apparatus requests the medical image management apparatus to transmit the test-object specifying information.

13. The medical image management system of claim 7, wherein
in the medical image generation apparatus, the image data generation section outputs transmission ultrasound wave toward the test object, receives reflective ultrasound wave from the test object to obtain a receiving signal, and creates ultrasound image data on the basis of the obtained receiving signal.

14. The medical image management system of claim 7, wherein
the control section of the medical image generation apparatus creates temporary test-object information when the test-object specifying information is not received from the information processing apparatus, and correlates the created temporary test-object information to the image data created by the image data generation section to store the temporary test-object information and the image data in a predetermined storage region, and
the medical image management system further includes an image data updating section to read out one (1) piece of the test-object information from the test-object information storage section and read out the image data stored in the predetermined storage region, and to update a content of the temporary test-object information of the read-out image data to the test-object information read out from the test-object information storage section.
